(19) United States
(12) Patent Application Publication
(10) Pub. No.: US 2018/0071014 A1
(43) Pub. Date: Mar. 15, 2018

(54) System and Methods for Irreversible Electroporation with Thermally Controlled Electrodes

(71) Applicant: ANGIODYNAMICS, INC., Latham, NY (US)

(72) Inventors: Robert Neal, Richmond, VA (US); Nahum S. Goldberg, Lawrence, NY (US); Gordon Single, Walnut Creek, CA (US)

(21) Appl. No.: 15/565,625

(22) PCT Filed: Apr. 11, 2016
(86) PCT No.: PCT/US16/26998

§ 371 Date: Oct. 10, 2017

Related U.S. Application Data
(60) Provisional application No. 62/145,581, filed on Apr. 10, 2015.
(60) Provisional application No. 62/151,513, filed on Apr. 23, 2015.
(60) Provisional application No. 62/173,538, filed on Jun. 10, 2015.

(51) Int. Cl. A61B 18/14; A61N 1/32; A61M 25/00; A61B 17/00

(52) U.S. Cl. CPC A61B 18/1477; A61N 1/327; A61B 2018/00613; A61B 17/00; A61M 25/00

(57) ABSTRACT

A treatment device and method for delivering electrical pulses capable of creating irreversible electroporation. The system may include a bipolar probe with open or closed perfusion with the purpose of controlling the electrical conductivity rise to eliminate electrical arcing, without significantly altering the electric field distribution and treatment zone. This invention may include perfusion together with the delivery of specific or customized pulse parameters to achieve clinically acceptable ablation sizes using a bipolar probe with while reducing the overall risk of arcing or system failure.

### CROSS REFERENCE TO RELATED APPLICATIONS

The present application incorporates by reference the entire disclosures of U.S. Provisional Patent Application 62/145,581, filed on Apr. 10, 2015; U.S. Provisional Patent Application 62/151,513, filed on Apr. 23, 2015; U.S. Provisional Patent Application 62,173/538, filed on Jun. 10, 2015; and U.S. Non-Provisional Patent Application 12/437,843, entitled Electroporation Device and Method of Use, filed May 8, 2009.

### BACKGROUND

Irreversible electroporation (IRE) and other electroporation-based therapies (EBTs), such as electrochemotherapy and electrogenetherapy, use the delivery of brief but intense electric pulses delivered into tissues through a number of electrodes to treat an intended treatment zone of tissue. These electric pulses subject cells in tissue to an electric field, which alters their native transmembrane potential, and at sufficient strength results in the creation of nanoscale defects that facilitate macromolecule transport and disruption of the membrane's ability to maintain cellular environment homeostasis. When the strength of the pulsing protocol is sufficient, the cell cannot recover from these defects and dies. EBTs encompass a range of therapeutic applications that exploit this phenomenon, particularly in regard to treatment of diseases in human or animal patients. The invention described herein is directed towards irreversible electroporation treatment; however it is conceivable to be applied to all types of EBTs.

The delivery of IRE pulses and the effect of these pulses on tissue has been previously described and documented, for example: U.S. Pat. No. 7,765,010, filed Feb. 6, 2006, entitled APPARATUS AND METHOD FOR TREATMENT OF BENIGN PROSTATIC HYPERPLASIA; U.S. Pat. No. 8,048,067, filed Dec. 21, 2004, entitled TISSUE ABLATION WITH IRREVERSIBLE ELECTROPORATION; U.S. Pat. No. 8,114,070, filed Jun. 24, 2005, entitled METHODS AND SYSTEMS FOR TREATING BPH USING ELECTROPORATION; U.S. Pat. No. 8,251,986, filed Jul. 10, 2009, entitled METHOD OF DESTROYING TISSUE CELLS BY ELECTROPORATION; U.S. Pat. No. 8,282,631, filed Sep. 20, 2011, entitled TISSUE ABLATION WITH IRREVERSIBLE ELECTROPORATION; U.S. Pat. No. 8,634,929, filed Jun. 22, 2010, entitled METHOD FOR TREATMENT OF NEOPLASTIC CELLS IN THE PROSTATE OF A PATIENT; and U.S. Pat. No. 9,078,665, filed Sep. 28, 2012, entitled MULTIPLE TREATMENT ZONE ABLATION PROBE; all of which are hereby incorporated by reference. The following reference is related to the subject matter of the present invention and incorporated herein in its entirety: U.S. Pat. No. No. 5,951,546, filed Sep. 30, 1997; entitled “ELECTROSURGICAL INSTRUMENT FOR TISSUE ABLATION, AN APPARATUS, AND A METHOD FOR PROVIDING A LESION IN DAMAGED AND DISEASED TISSUE FROM A MAMMAL”.

Current state of the art commercially available IRE treatment devices do not use perfusion or other cooling fluid to control the temperature of the tissue or probe within the treatment site. While the possibility of using IRE and other EBTs has been well known as a means to mitigate thermal damage during a treatment, the actual effect on treatment outcome in terms of ablation size and damage due to thermal effects has only been recently discussed in the literature, for example Davalos, et al, “IMPLICATIONS AND CONSIDERATIONS OF THERMAL EFFECTS WHEN APPLYING IRREVERSIBLE ELECTROPORATION TISSUE ABLATION THERAPY” The Prostate, published by Wiley Periodicals, Inc.; 2015. As will be discussed in more detail below, this invention discloses the use of perfusion combined with other key novel aspects of the system to solve problems associated with the commercially available IRE treatments options.

Additional concerns and/or challenges with the current IRE treatment devices known and used in the art may include: electrode arcing potential during a treatment resulting in a system crash/failure leading to lengthening of overall procedure time and/or inability to complete the procedure; unintended rise in temperature adjacent to the probe; restriction on certain probe placements due to complexity; difficulty in aligning two or more monopolar probes on a parallel axis and maintaining consistent probe insertion depth; tight tolerances required in spacing of multiple probe electrodes; difficulty in measuring the size of the treatment site when determining treatment parameters; requirement to navigate around anatomical obstacles (such as bone, spleen, or other non-target tissue); and unintentional flexing of the probe shaft during placement in the treatment site, resulting in misalignment of electrodes relative to each other.

Another problem with current commercially available IRE treatment systems is the high number of total pulses delivered for IRE therapies which, depending on the patient and tissue conditions, may result in significant cumulative and undesirable thermal effects. For example, some pieces of literature in the art, for example Wagstaff P G K, et al, “IRREVERSIBLE ELECTROPORATION OF THE PORCINE KIDNEY: TEMPERATURE DEVELOPMENT AND DISTRIBUTION” Elsevier; 2014, reported that currently accepted IRE treatment parameters may result in temperature levels as high as 59° C. in regions between several pulse pairings at even typical pulse protocols. In addition to thermal damage which may mitigate or eliminate the benefits of IRE as a non-thermal procedure, an unintended temperature rise may also change the properties of the tissue and thus alter treatment outcome. In addition, thermal issues may add to arcing potential when applying desired treatment parameters.

A key advantage of the system described herein over other currently known IRE treatment devices includes the use of perfusion together with the delivery of a specific or customized pulse parameters to achieve clinically acceptable ablation sizes using a single bipolar probe while reducing the overall risk of arcing and overall procedure time. For example, a single stick bipolar probe of this invention can be used to create the same clinically acceptable ablation sizes compared to multiple monopolar probes.

### FIELD OF THE INVENTION

The disclosure generally relates to the systems and method for delivery of electrical pulses to treat an intended treatment site. The system may also comprise of a bipolar probe with a perfusion system and ability to control pulse parameters.

### SUMMARY OF THE DISCLOSURE

This disclosure is based upon the concept of temperature control for IRE therapies to improve treatment outcome. The intentions of this invention include eliminating possible thermal damage or the effects of creating a thermal zone near the treatment site, improving pulse stability, reducing arc potential, and facilitating larger IRE ablation zones by permitting greater voltages and larger total pulse energy protocols, without causing a significant increase in targeted tissue temperature. An additional advantage is confirmation and demonstration of reducing the extent of thermally affected tissues as a result of perfusion. Reducing or eliminating the extent of thermal damage improves IRE's morbidity profile in therapeutic applications and further ensures that the bulk of ablated tissue does not include thermal damage to critical sensitive structures such as blood vessels, neurovascular bundles, or ductal systems.

In one aspect of the invention, a medical device is provided for ablating tissue cells in a treatment region by irreversible electroporation without thermally damaging the tissue cells, comprising: a temperature controlled perfusate; an electrode probe having a perfusate channel for receiving the temperature controlling perfusate and at least two electrodes adapted to apply irreversible electroporation (IRE) pulses to the tissue cells in the treatment region; a control device for controlling the IRE pulses to the at least two electrodes and operable to provide the temperature controlling perfusate to the perfusate channel of the probe to maintain the temperature of the targeted tissue cells between 20 degrees Celsius and 50 degrees Celsius.

In one aspect of the invention, a medical device is provided wherein a temperature controlling perfusate controls an electrical conductivity rise in the tissue cells sufficiently to eliminate electrical arcing, but without significantly altering the electric field distribution and treatment zone.

In one aspect of the invention, a medical device is provided wherein a control device provides the temperature controlling perfusate to the perfusate channel to maintain the temperature of the tissue cells at between 30 degrees Celsius and 45 degrees Celsius.

In one aspect of the invention, a medical device is provided wherein an electrode probe includes a temperature sensor that measures the temperature of the targeted tissue cells and the control circuit adjusts the amount of the temperature controlling perfusate to the perfusate channel in real-time based on the monitored temperature.

In one aspect of the invention, a medical device is provided comprising a power distribution unit.

In one aspect of the invention, a medical device is provided comprising a pump coupled to the control device, wherein the control device controls the pump to vary the flow rate of the temperature controlling perfusate.

In one aspect of the invention, a medical device is provided comprising a pulse generator capable of generating the IRE pulses wherein the IRE pulses in one train between the two electrodes has a first polarity and the IRE pulses in an adjacent train has a second polarity opposite from the first polarity.

In one aspect of the invention, a medical device is provided wherein a control device monitors the current flow through the at least one electrode and provides the temperature controlling perfusate to the perfusate channel based on the monitored current flow.

In one aspect of the invention, a medical device is provided wherein a control device monitors the current flow through the at least one electrode and provides the temperature controlling perfusate to the perfusate channel based on the rate of change of the monitored current.

In one aspect of the invention, a medical device is provided wherein an electrode probe includes fluid ports along its distal end, wherein the temperature controlling perfusate is injected into the target tissue cells through the fluid ports.

In one aspect of the invention, a medical device is provided wherein a control device calculates tissue conductivity based on the current flow through the at least one electrode.

In one aspect of the invention, a medical device is provided wherein a control device applies a test pulse through the electrode and calculates the tissue conductivity based on the current flow from the applied test pulse.

In one aspect of the invention, a medical device is provided comprising a temperature sensor that senses the temperature of the target region, and a control device which calculates tissue conductivity based on the sensed temperature.

In one aspect of the invention, a medical device is provided wherein a control device controls the flow of the temperature controlling perfusate through the perfusate channel based on the number of IRE signals, current or the amount of power applied to the target region.

In one aspect of the invention, a medical device is provided comprising a memory that stores at least one electrical parameter for a plurality of tissue types and the control device controls the flow of the temperature controlling perfusate through the perfusate channel based on the at least one electrical parameter for the type of tissue cells being treated.

In one aspect of the invention, a medical device is provided comprising: a pumping device that controls the flow rate of the temperature controlling perfusate through an source tube and return tube; wherein the pumping device is controlled by the control unit.

In one aspect of the invention, a medical method is provided for ablating tissue cells in a treatment region by irreversible electroporation without thermally damaging the tissue cells, comprising: applying irreversible electroporation (IRE) signals to the tissue cells in the treatment region through at least one electrode of an electrode probe; providing a temperature controlling perfusate to a perfusate channel of the electrode probe to maintain the temperature of the tissue cells at either 5 degrees Celsius or greater or 50 degrees Celsius or less.

In one aspect of the invention, a medical method is provided for ablating tissue cells in a treatment region by irreversible electroporation without thermally damaging the tissue cells, comprising: applying irreversible electroporation (IRE) signals to the tissue cells in the treatment region through at least one electrode of an electrode probe; providing a temperature controlling perfusate to a perfusate channel of the electrode probe to maintain the temperature of the tissue cells at 45 degrees Celsius or less.

In one aspect of the invention, a medical method is provided comprising a method of ablating tissue cells in a treatment region by irreversible electroporation without thermally damaging the tissue cells wherein the step of providing includes providing the to a controlling perfusate to the perfusate channel to maintain the temperature of the target tissue cells at body temperature.

In one aspect of the invention, a medical method is provided comprising a method of ablating tissue cells in a treatment region by irreversible electroporation without thermally damaging the tissue cells further comprising: controlling an electrical conductivity rise in the tissue cells sufficiently to eliminate electrical arcing with the temperature controlling perfusate; the step of eliminating electrical arcing significantly altering the electric field distribution.

In one aspect of the invention, a medical device is provided for ablating tissue cells in a treatment region by irreversible electroporation without thermally damaging the tissue cells, comprising: an electrode probe having first and second spaced apart electrodes; a pulse generator that generates IRE pulses as follows: a first row of pulses consisting of a first pulse train and a second pulse train, the first pulse train consisting of at least five individual pulses, the first pulse train having a first polarity, an inter-train delay of at least 2 second, the second pulse train consisting of at least five individual pulses, the second pulse train having a second polarity that is the opposite of the first polarity, an inter-row delay of up to at least 10 second, and a second row of pulses consisting of a third pulse train and a fourth pulse train.

### BRIEF DESCRIPTION OF THE DRAWINGS

FIG. 1 shows a functional block diagram of an electroporation system contemplated for the current invention.

FIG. 2 shows a perspective view of one embodiment of the probe.

FIG. 3A shows an exploded view of the probe.

FIG. 3B-FIG. 3H show partial side cross-sectional views of the probe shaft components at different stages of assembly.

FIG. 4 shows a partial side view of the distal end of the probe.

FIG. 5 depicts a partial side view of the distal end of the probe with zone of arcing potential.

FIG. 6 shows a partial side view of the distal end of the probe with the predicted ablation zone.

FIG. 7 shows a partial perspective view of the perfusion system.

FIG. 8 shows a partial perspective view of the hub of the perfusion system.

FIG. 9 shows a partial perspective cross-sectional view of the hub of the perfusion system.

FIG. 10A shows a partial cross-sectional side view of the probe handle.

FIG. 10B shows a partial perspective cross-sectional view of the distal end of the probe.

FIG. 10C shows a side cross-sectional view of the probe.

FIG. 10D shows a partial side cross-sectional view of fluid path within the probe handle.

FIG. 11 depicts a side view of the distal region of another embodiment of the probe.

FIG. 12 depicts a partial cross-sectional side view of the distal regions of yet another embodiment of the probe.

FIG. 13 depicts a functional block diagram of yet another embodiment of an electroporation system contemplated of the current invention.

FIG. 14 depicts a schematic representation of the power distribution unit with controller and generator interfaces.

FIG. 15 shows a table of a simulation using research results depicting ablation volumes at varying temperatures contemplated for the current invention.

FIG. 16 shows a line chart of a simulation using volume of temperature exposure versus temperature threshold for varying perfusate temperatures contemplated for the current invention.

FIG. 17 shows a line chart of a simulation using volumes of exposure versus perfusate temperature at multiple temperature thresholds contemplated for the current invention.

FIG. 18 shows a chart of specific parameters of IRE energy pulse delivery contemplated for the current invention.

FIG. 19 depicts a flowchart showing the method of IRE delivery contemplated for the current invention.

### DETAILED DESCRIPTION OF THE INVENTION

The present invention can be understood more readily by reference to the following detailed description and the examples included therein and to the figures and their previous and following description. The drawings, which are not necessarily to scale, depict selected preferred embodiments and are not intended to limit the scope of the invention. The detailed description illustrates by way of example, not by way of limitation, the principles of the invention.

The skilled artisan will readily appreciate that the devices and methods described herein are merely exemplary and that variations can be made without departing from the spirit and scope of the invention. It is also to be understood that the terminology used herein is for the purpose of describing particular embodiments only and is not intended to be limiting.

As used herein, the term “proximal” denotes the direction closer to the operator and the term “distal” denotes the direction closer to (inserted into) the patient.

As used herein, the term “perfusate” means a non-corrisive, sterile physiologic fluid such as distilled water, saline solution, buffer solution, gas (such as CO2) such as a dextrose buffer, or LRS (lactated ringer's solution), Hartmann's solution or any combination thereof. The term “perfusion” means circulating or pumping the perfusate so that the perfusate enters into the fluid channels within the probe and circulating or pumping the perfusate through the probe such that the perfusate is injected, infused or enters into the tissue within the treatment zone. Perfusion may also include controlling the temperature or conductivity of the perfusate, controlling to internal and surrounding temperatures of the probe, and infusion of the perfusate into the tissue such that the perfusate interacts with the cells of the tissue within the treatment zone.

As disclosed herein, the reference to “electrodes” may include physically discrete components that serve to deliver the electric pulses, but it can also indicate individual energized surface components within a single device, such as a bipolar electrode or an electrode with individually energizable surfaces, such as electrically isolated tines or conducting wires in a tubular catheter-style device. The latter style of electrodes would particularly benefit from being able to fine tune control of the pulse delivery, and can often times include more than six individual surfaces in which to deliver the electric pulses. The electrodes of this invention may also be used together with a grounding pad. In one embodiment, the grounding pad may be intended to be placed on the surface of the treatment tissue while an electrode is inserted into or adjacent to the treatment tissue.

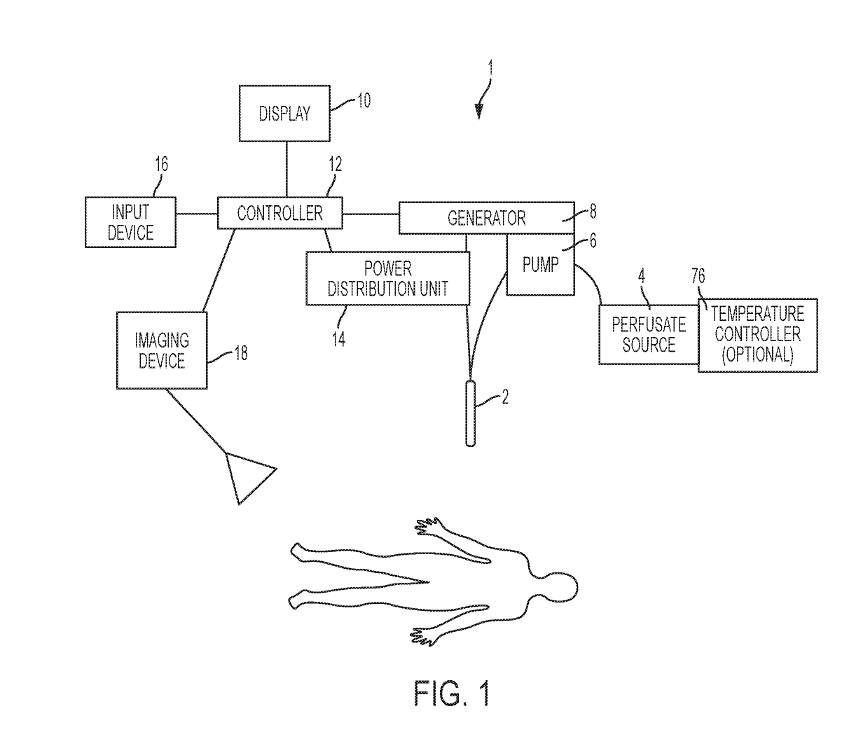

Referring now to FIG. 1, the system 1 of this disclosure may include, but is not limited to, the following elements: a disposable probe 2, a perfusate source 4, a pump 6, an optional temperature controller 76, a generator 8, a display 10, a controller 12, a power distribution unit 14, an input device 16, and an imaging device 18. These various components are designed to work together and be integrated into a single treatment system. The treatment system 1 is designed to be used to perform an irreversible electroporation procedure, however it is possible that the system 1 may be used for other EBTs. While not all components of this system 1 or kit may be packaged, shipped, or sold together, it is still understood that the various components will work together as a single system 1. For example, it may be common for the imaging device 18 to be used with this system 1 to be an ultrasound device, MRI system, or another commonly known imaging device that is off the shelf, or otherwise already used in a hospital setting. However, the system may be designed such that it can incorporate such an imaging device 18 into the system 1, or alternatively it can interface with the controller 12, such that the information or feedback received from the imaging device 18 may be used by the user of system 1.

The system 1 may also include one or more probes 2. The probe 2 may be operably connected to a pump 6 and also connected to a power distribution unit 14 and/or a generator 8. The probe 2 is intended to deliver therapeutic energy to the patient. In one embodiment, the probe 2 is designed to be inserted into the patient's body such that the probe 2 is within the desired treatment site. Alternatively, the probe 2 may be placed on the outside surface of the patient's body. The probe 2 of this system may include, but is not limited to, a bipolar probe having at least two electrodes on the probe 2, multiple monopolar probes having at least one electrode, or a single monopolar probe having at least one electrode on the probe 2 for use with a grounding pad placed externally on the patient's skin. A perfusate source 4 may provide perfusate fluid through the pump 6 to the probe 2. A computer including a user display 10, input device such as a keyboard 16, and controller 12 may be used to input instructions and/or treatment parameters which are transmitted to generator 8/power distribution unit 14 to generate specific pulse trains to the probe 2. An optional imaging device 18, used to visualize the treatment area prior to, during and/or after the pulse delivery, may be separate or integrated with the system. An optional temperature control unit 76 is in communication with the probes 2 via a thermocouple or other sensing component to monitor temperature in and/or around the probe, and allow automatic or manual adjustment to the generator 8 parameters and/or perfusion flow rate based on the temperature monitoring.

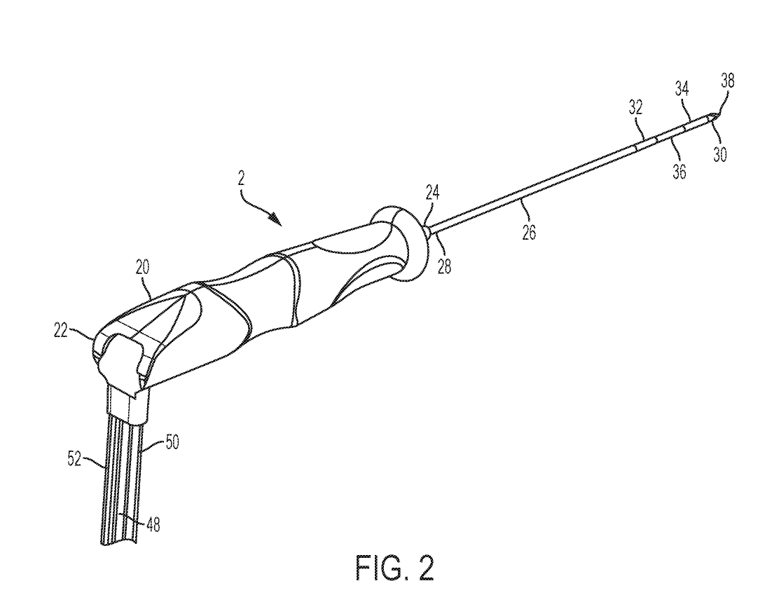

Referring now to FIGS. 2-5, one embodiment of this invention includes a bipolar probe 2. A major advantage of the bipolar probe 2 of this system is the ease of use during placement of the probe 2 prior to treatment, compared with placement of multiple monopolar probes. Because the probe 2 is bipolar and incorporates at least two electrodes 32, 34, the user only needs to place a single probe 2 into the intended treatment site in order to achieve clinically useful ablation volumes, compared to commonly known commercial IRE devices in which require placement of multiple monopolar probes. The bipolar probe 2 combined with the specific pulse parameters and infusion of perfusate, both described in more detail below, result in more clinically useful treatment outcome with larger ablation zones.

Proper probe placement is a key aspect of a successful IRE procedure. The user must make a decision as to the best placement for the probes relative to the treatment site. A current commercially available treatment typically requires anywhere from two to six monopolar probes to be placed in the patient. These monopolar probes have a shaft extending from the handle to a distal end section where a single monopolar electrode is position. These single monopolar probes are commonly placed with a gap of at least 1 cm and up to 2.5 cm between each probe. Furthermore, each monopolar probe may have up to a 2 cm active electrode exposure length. A problem currently exists in the art related to the complexity and precision required when placing these monopolar probes, specifically related to the current systems required for alignment of multiple probes relative to each other along an x-axis, y-axis, and z-axis. It is common for a user to spend significant amounts of time planning proper probe placement and then accurately placing the probes based on the planned location before the treatment can even begin. Moreover, ensuring precise probe placement is critical for a successful and complete IRE treatment session. For example, if the single monopolar probe is mispositioned or misdeployed relative to planned placement and/or misaligned relative to another monopolar probe, this may lead to potential complications including unpredictable ablation zones; unknown treatment outcomes; and a greater potential for arcing which may lead to system failures. Thus, proper probe placement of multiple probes is one of the most significant clinical challenges today when conducting an IRE procedure.

This invention solves a need in the art to simplify probe placements, thereby reducing treatment times and potential for unintended complications, saving the user and hospital time and money, and benefiting the patient by increasing the likelihood of a successful treatment. One of the primary benefit of only having to place a single bipolar probe 2 within the treatment site is a reduction in the overall time required for probe placement planning and actual positioning, thereby reducing overall procedure time which saves the doctor/hospital money and reduces unwanted or unintended risks associated with anesthesia to the patient. Additionally, the use a bipolar probe 2 does not require alignment of multiple monopolar probes in a parallel arrangement which is often difficult to achieve, leading to incomplete or unintended ablation zones. A single bipolar probe is also advantageous in that only a single puncture is needed, less imaging is required, as well as providing the user with more flexibility in probe placement around bone or other non-targeted structures. The bipolar probe 2 of this system, combined with the treatment parameters and treatment method described below has been shown to create a more predictable, consistent, and larger ablation zone compared ablations created using multiple monopolar probes. The use of a single bipolar probe 2 provides more predictability in the geometry of the delivery device thereby simplifying the pulse parameter selection and allows for tight ablation dimension tolerances to be achievable.

Referring now to FIG. 2, the probe 2 assembly may include a handle 20 having a proximal end 22 and a distal end 24, an elongated probe body 26 that extends from the distal end 24 of the handle towards the treatment zone. The probe body 26 has a proximal end 28, which extends into the handle 20 for a selected distance, and a distal end 30. The probe 2 further comprises at least two electrodes 32, 34 near the distal end 30 of the probe body 26. These at least two electrodes 32, 34 are designed such that they are in a spaced relationship with one another along the probe body 26 with an insulated spacer 36 positioned between electrodes 32, 34. The probe 2 may also include a distal tip 38 which is capable of piercing through skin and other tissue such that the probe may be percutaneously or interoperatively placed in the treatment zone. The distal tip 38 may be made of a non-conductive material, or alternatively in some embodiments may be made of a conductive material and act as an electrode.

The electrodes 32, 34 of the probe 2 may be designed such that they are independently activated electrodes on the surface of the probe body 26. Each electrode 32, 34 may be capable of switching between a positive polarity and a negative polarity during a single IRE treatment.

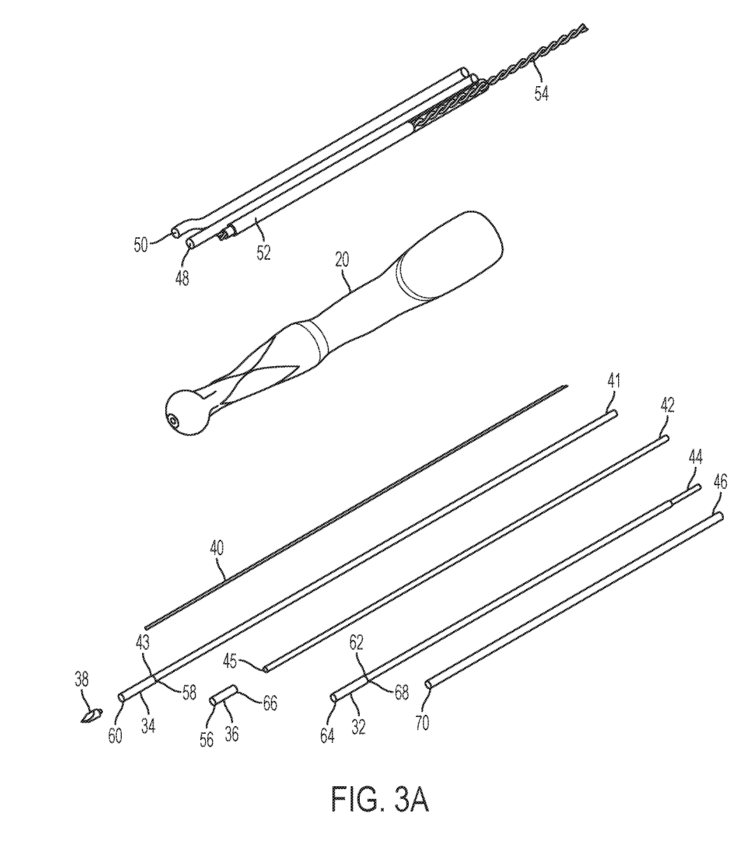

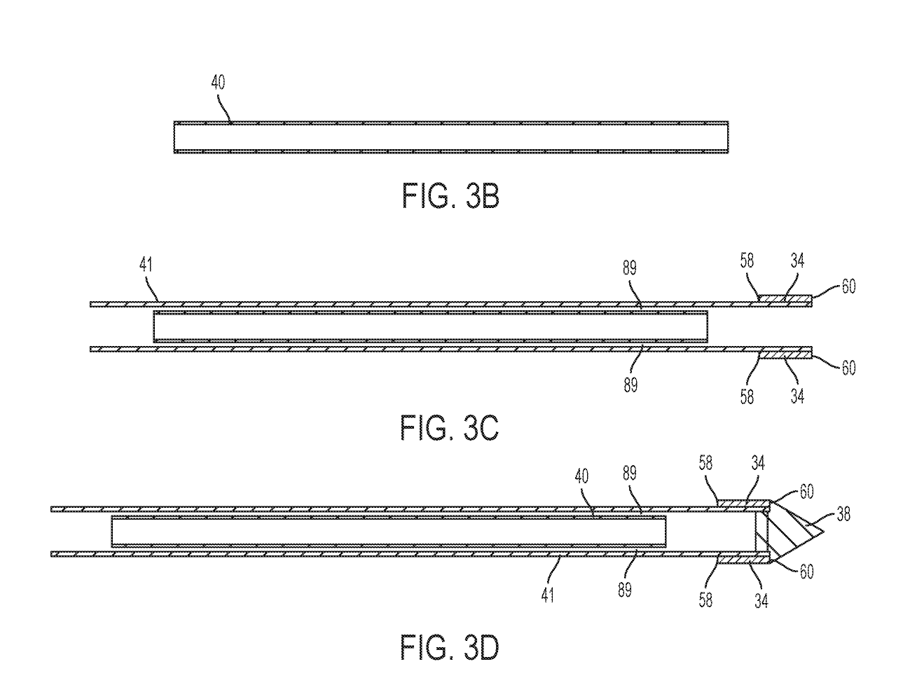

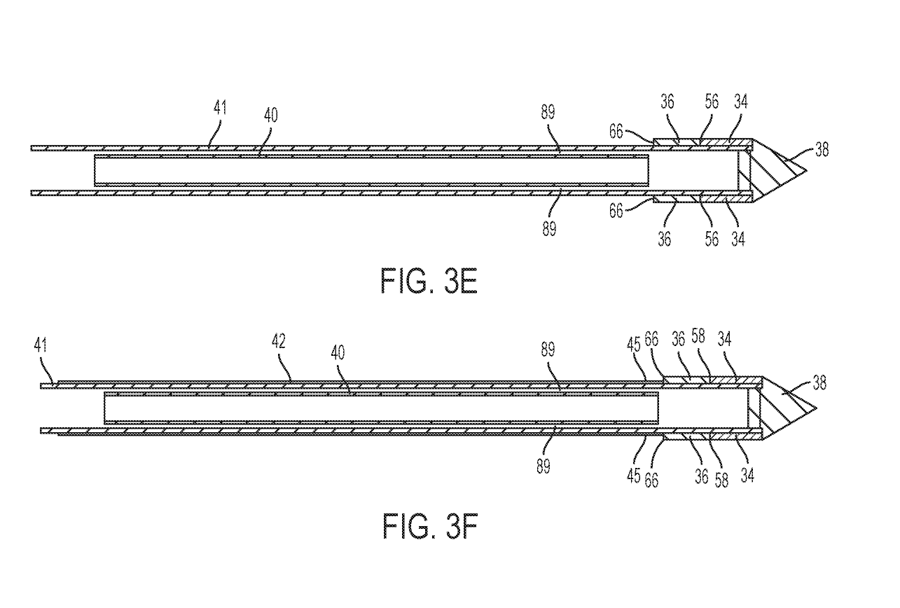

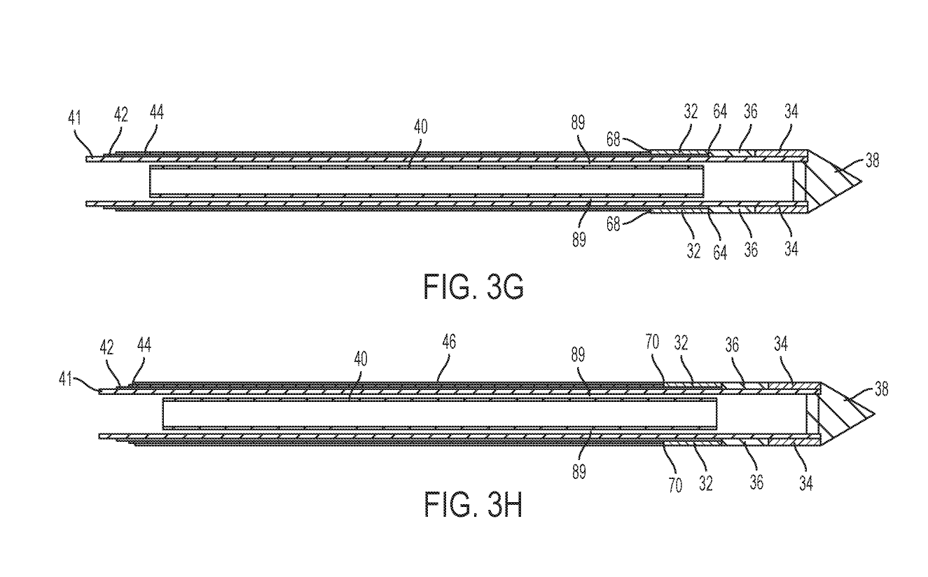

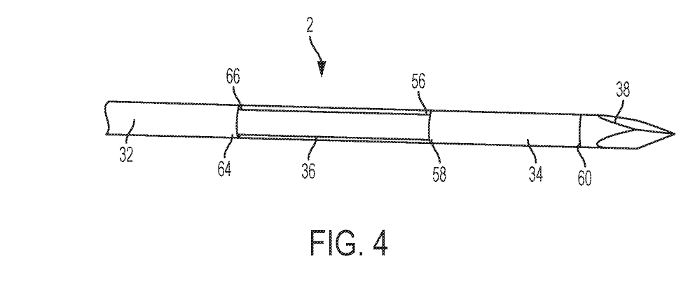

Referring now to FIG. 3A-FIG. 4, additional components of the probe 2 may include a perfusate channel 40, a first conductor tube 41, a first electrode 34, a first insulator tube 42, a spacer element 36, a second conductor tube 44, a second electrode 32, a second insulator tube 46, a distal tip 38, a first perfusate tube 48, a second perfusate tube 50, a power cable tube 52, and a power cable 54. One embodiment of manufacturing the probe body 26 is shown in FIGS. 3B-3H.

Referring first to FIGS. 3B-3D, the perfusate channel 40 is coaxially placed inside of the first conductor tube 41. The empty space between the outer wall of the perfusate channel 40 and the inner wall of the first conductor tube 41 comprises the coaxial return lumen 89. The first electrode 34 is securely attached to the distal end of the first conductor tube 41 by welding, adhesive, or other known techniques in the art. Next, the distal tip 38 is securely attached to the distal most end of the first conductor tube 41 by an interference fit, welding, adhesive, or other known techniques in the art. The distal tip 38 is connected to the first conductor tube 41 such that there is a fluid tight connection to prevent any perfusate from escaping out the distal most end of the first conductor tube 41.

Referring next to FIG. 3E-3F, a spacer 36 is coaxially placed over the first conductor tube 41. The distal most end 56 of the spacer 36 abuts against the proximal most end 58 of the first electrode 34. The first insulator tube 42 is then coaxially placed over the first conductor tube 41 such that the distal most end 45 of the insulator tube 42 abuts up against the proximal most end 66 of the spacer 36. As shown in FIG. 3G-3H, the second conductor tube 44 is then coaxially placed over the first insulator tube 42. The second electrode 32 is securely attached to the distal most end of the second conductor tube 44 by welding, adhesive, or other known techniques in the art. The distal most end 64 of the second electrode 32 abuts up against the proximate most end 66 of the spacer 36. Lastly, the second insulator tube 46 is coaxially placed over the second conductor 44 such that the distal most end 70 of the second insulator tube 46 abuts up against the proximate most end 68 of the second electrode 32.

The probe 2 may be designed to have a relatively constant outer diameter along the shaft length such that there is a smooth transition between the outer wall of the second insulator tube 46, the second electrode 32, the spacer 36, the first electrode 34 and the proximal portion of distal tip 38, as shown in FIG. 4. The purpose of this smooth transition is so that the probe may have an easy insertion through tissue during placement. A temperature sensor may be placed at any place along the probe body, such as near the distal end near the first electrode, the second electrode, or the spacer.

The perfusate channel 40 may be made from such material as stainless steel or other non-corrosive metals or rigid materials. The first 41 and second conductor tubes 44 may be made from such materials as stainless steel or other non-corrosive metals or rigid materials. The first 42 and second insulator tubes 46 may be made from such materials as polyimide, heat shrink, or other electrically insulating materials. The spacer 36 may be made from such material as PEEK plastic, ceramic, or other rigid electrically insulating materials. The distal tip 38 may be made from such material as PEEK plastic, ceramic, or other rigid electrically insulating materials. In an alternative embodiment, the distal tip 38 may be comprised of a conductive material, if tip 38 is intended to act as one of the electrodes. The first 48 and second 50 perfusate tubes may be made from such materials as PVC, PTFE, or other flexible biocompatible polymer tubing.

It is the intention of this system and method described herein to solve problems associated with undesirable thermal effects when delivering IRE treatment. The present invention achieves an optimal balance of (1) creating the largest ablation volume possible against (2) maintaining a threshold temperature in the target area which ensures that no thermal damage occurs, especially in those tissue areas adjacent to the active electrodes where tissue desiccation is possible. By maintaining this balance between ablation volume and temperature, the system is less likely to generate arcing conditions at the electrodes. In one embodiment the present invention uses perfusion to control the temperature of the tissue immediately surrounding the electrodes within a relatively narrow range of 20-45° C., specifically 30-40° C. The upper limit of this controlled temperature range eliminates the possibility of thermal damage to the tissue and other cellular structures, and reduces arcing or sparking between electrodes, while the lower limit of this controlled temperature range ensures maximum ablation volume at the lower tissue temperatures.

Advantages of the perfusion of this system include mitigating the extent of unintended tissue thermal damage when delivering IRE pulses and preventing arcing between electrodes and the resultant generator faulting. Perfused probes result in a reduction in the bulk tissue temperature rises and maximum temperatures proximate to the electrodes. This dramatically reduces the extent of unintended thermal damage, including the variety of thermal damage that risks morbidity to the sensitive cellular structures.

One definition of arcing may include a discharge of matter between two electrodes that is caused by electrical current ionizing gasses. Arcing occurs when a medium of high resistance, low conductivity, exists in the path that current would normally flow, which is along the path of least resistance. One possible reason for arcing during an IRE procedure may be the result of ionic movement towards the positively charged electrode. Ions within the soft tissues are negatively charged or positively charged. Negatively charged ions will flow towards the positively charged electrode(s) during the IRE procedure, thus potentially leaving behind an empty space or an air gap, at the negatively charged electrode. Positively charged ions will flow towards the negatively charged electrode(s) during the IRE procedure, thus potentially leaving behind an empty space or air gap, at the positively charged electrode. If more negatively charged ions are present in the tissue, there could be a greater possibility for air pockets to form nearest the negatively charged electrode. Air pockets increase resistance thus contributes to likelihood of arcing.

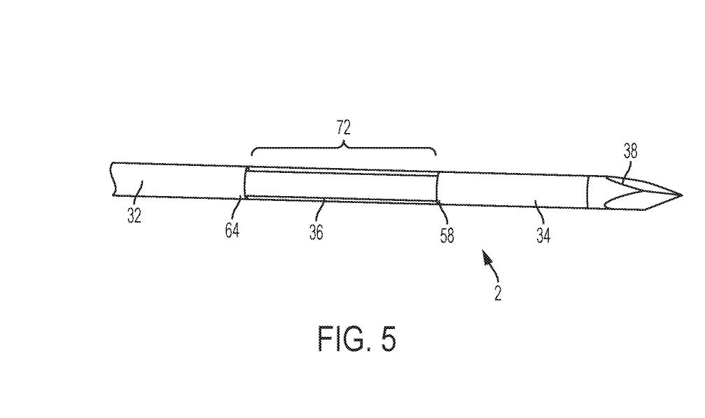

If arcing occurs during an IRE procedure it commonly occurs at the shortest distances between each electrode, or said another way, it will occur where the electrodes are closest together because this is the path of least resistance. As shown in FIG. 5, the bipolar probe of this invention would have the highest occurrence of arcing at the distance 72 between the distal most end 64 of the first electrode 32 and the proximal most end 58 of the second electrode 34. To mitigate such a risk of arcing at this distance 72, this system uses: (i) perfusion for temperature control at distance 72 along probe 2 with greatest risk of arcing; in combination with (ii) a specific pulse parameter setting that alternates polarity of the electrodes 32, 34 throughout a single IRE treatment, which will be discussed in more detail below. Alternating polarity may reduce to potential charge in the tissue thereby reducing the arcing potential.

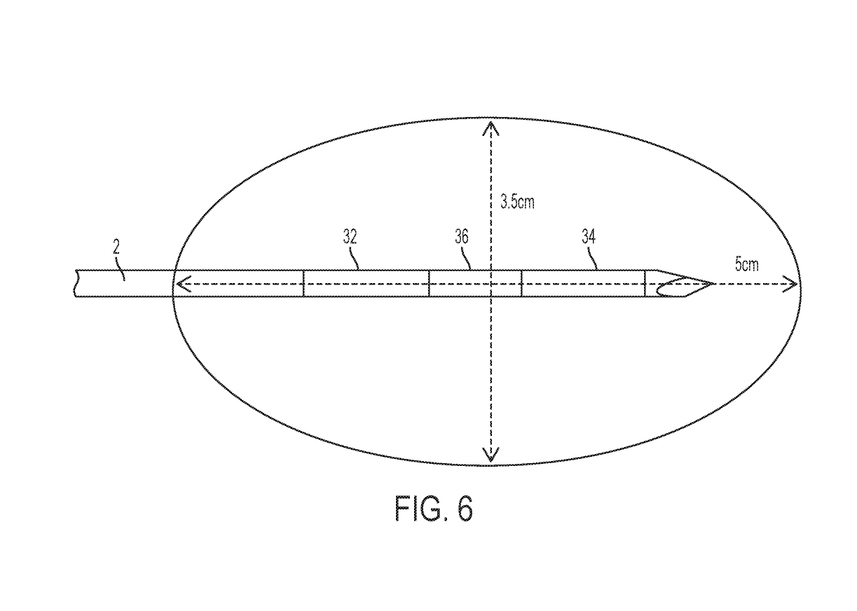

Perfusion prevents or reduces the likelihood of the generator from arcing or faulting by improving pulse stability and reducing electrical current and arcing incidence. While electric current and arcing are often correlated, they are in fact two different modes by which the electroporation generator can fail. A reduction in voltage delivered to the tissue also reduces the power being delivered to the tissue, thereby reducing the likelihood of arcing potential. Perfused electrodes address both of these failure modes while increasing the voltages that can be delivered to achieve a larger ablation or treatment zone. The probe 2 of this system 1 may include a bipolar electrode probe 2 that, when used together with the system 1 and the method described below, can consistently attain clinically useful ablation dimensions with reduced/eliminated arcing. Clinically useful ablation dimensions will vary based on tumor modality and location. As a non-limiting example, a clinically useful ablation size for a typical liver tumor may be the size of greater than 3 cm, but may include the specific treatment zone of at least 5 cm by 3.5 cm, as shown in FIG. 6, which is equivalent to the ablation zone achievable in current commercially available IRE device which uses at least two monopolar probes.

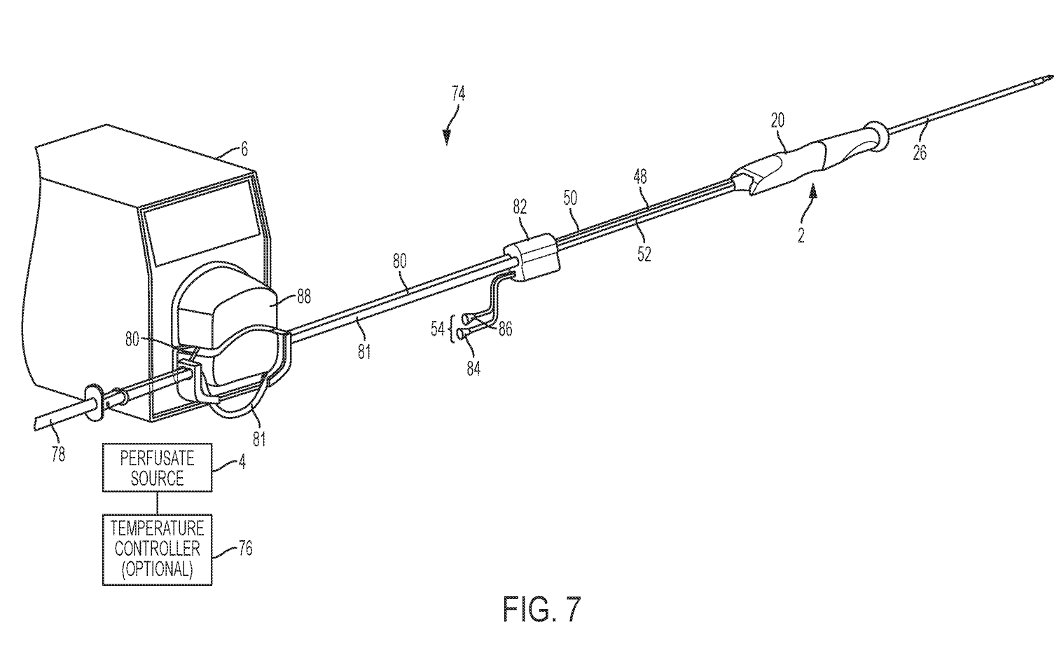

Referring now to FIG. 7-FIG. 12, the perfusate system 74 will be described in detail. The perfusate system 74 may be comprised of a perfusate source 4, an optional temperature control unit 76, a pump 6, a fluid spike 78 or other attachment for connecting to a perfusate source 4, a source perfusate tube 80, a return perfusate tube 81, a hub 82, a first perfusate tube 48, and a second perfusate tube 50. The purpose of the perfusate system 74 is to control the temperature of the probe 2 and/or tissue within the treatment zone. Temperature control has been found to correlate with arcing potential, so it is within the scope of this invention to prevent potential arcing of the system by using the perfusate system 74 to control the temperature of the probe 2 and treatment site during an IRE procedure. Various components of the perfusate system 74 may be multi-use such that they can be used for different patients, such as the pump 6, temperature controller 76, and even the perfusate source 4 in certain embodiments. The perfusate system 74 may require a priming sequence that can be controlled by the GUI and/or controller 12.

As will be discussed in more detail below, the temperature of the perfusate may vary depending on the type of IRE treatment being performed, the type of tissue to be treated, or the specific pulse parameters to be used. The system may optionally include a temperature controller 76 that is in communication with the system controller 12 which may monitor the temperature levels of the perfusate, probe and/or surrounding tissue, and automatically adjust temperature levels to minimize arcing potential which simultaneously maximizes tissue ablation volumes. The temperature controller 76 may also heat up the perfusate to body temperature, maintain the perfusate temperature at room temperature, and/or cool the perfusate to any temperature above freezing. Conversely, if the system is to use room temperature perfusate then the temperature controller 76 may not be a required part of the perfusate system 74.

The pump 6 may include any number of commercially available pumps that are commonly known in the art such as a peristaltic pump, centrifugal pump, roller pump, piston driven pump, or other known pumping mechanisms. One advantage of this system is its compact footprint. The purpose of this compact design is to allow the users the greatest amount of flexibility when it comes to storing, using, and moving this system. Because this system is intended to be functional and compact, one embodiment of this system is for the pump 6 is assembled together with the generator 8 into a single housing shown). Such a design would achieve a compact single box or control unit that could easily be moved and would not take up a large footprint inside the hospital or clinical setting. For example, such an integrated pump and generator system is described in U.S. Provisional Patent Application No. 62/238,299, filed Oct. 7, 2015, hereby incorporated by reference.

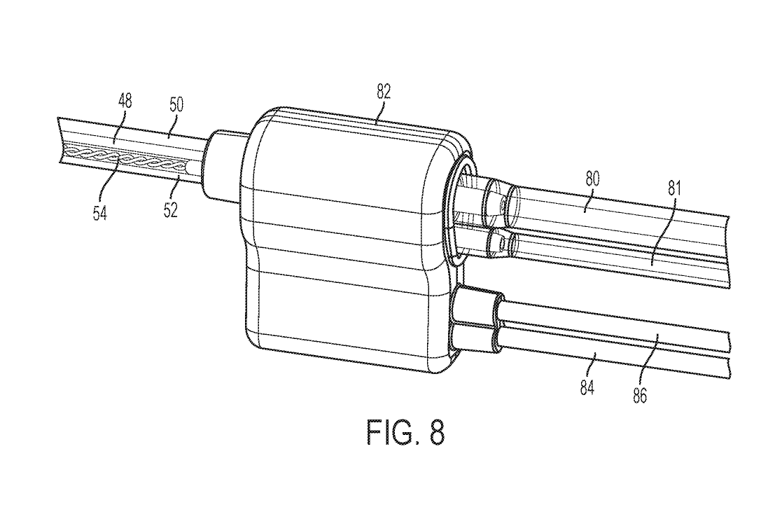

As shown in FIG. 8, the hub 82 is a junction point in which the source perfusate tube 80, the return perfusate tube 81, and the power cables 54 transition into the first perfusate tube 48, the second perfusate tube 50, and the power cable tube 52. The purpose of the hub 82 is to increase usability and user efficiency of the system. In one embodiment the power cable tube 52, the first perfusate tube 48 and second perfusate tube 50 are connected or joined together during manufacturing, thereby eliminating multiple loose cables and tubing extending probe proximal end of the handle 20. The power cables 54 may be comprised of a first power cable 84 and a second power cable 86. The first power cable 84 may be connected to the generator 8 or other power source and provides a conduit for the flow of electrical current to the first electrode. The second power cable 86 may be connected to the generator 8 or other power source and provides a conduit for the flow of electrical current to the second electrode. The first power cable 84 and the second power cable 86 may be aligned within hub 82 so that both power cables 54 extend within and co-axially along the power cable tube 52. The source perfusate tube 80 is in fluid communication with the spike 78 and the perfusate source 4. The source perfusate tube 80 will be placed within the pump head 88. In one embodiment, the pump 6 is a peristaltic pump as known in the art and the source perfusate tube 80 is aligned on top of the rollers of the pump head 88. In this embodiment, the return perfusate tube 81 would not be aligned within the pump head 88 but rather routed around the pump head 88, as shown in FIG. 7, and back to either a waste container (not shown) or the perfusate source so that used perfusate be reused.

Within the hub 82 the source perfusate tube 80 is aligned with and connected to the first perfusate tube 48 so that the source perfusate tube 80 is in fluid communication with the first perfusate tube 48. Also within the hub 82 the second perfusate tube 50 is aligned with and connected to the return perfusate tube 81 so that the second perfusate tube 50 and return perfusate tube 81 are in fluid communication. The first power cable 84 and the second power cable 86 may be twisted, combined, or otherwise connected together to form the power cables 54 that extend within the power cable tube 52.

The flow of perfusate within this system will depend on if it is an “open” system or a “closed” system. For example, a “closed” system is one which the perfusate only circulates inside of the probe and does not enter into the tissue within the treatment zone. Conversely, an “open” system refers to a perfusion system in which the perfusate is directly injected or infused into the tissue within the treatment zone. Embodiments of both an “open” and “closed” system are described in more detail below.

Referring now to FIGS. 7-10D), the first embodiment is a closed system in which the perfusate is circulated within the probe body and is not introduced into the surrounding tissue. The purpose of a closed perfusate system in which the perfusate is circulated and contained within the probe 2 is so the continuous circulation of perfusate may control the temperature of the probe 2 and/or electrodes 32, 34 at the hottest or highest temperature points, thereby reducing sudden current inflections and/or arcing. Therefore, the temperature of the perfusate used with the closed system will directly control the temperature at which the probe 2 is intended to be maintained.

In one embodiment, the flow of the perfusate is contained within the probe 2 and designed to be a closed system. The probe 2 may be manufactured with the first perfusate tube 48 and second perfusate tube 50 already assembled with the handle 20 so that the user does not need to make any fluid connections between the handle 20 and the perfusate tubing 48, 50. The flow of perfusate in the closed system of this embodiment originates with the perfusate source 4. The perfusate source 4 may be a bag of normal saline or any other perfusate. First, the user may prime the perfusate system by placing the spike 78 in the perfusate source 4 to begin the flow of perfusate. The source perfusate tubing 80 may then be placed on the pump head 88. The return perfusate tubing 81 may be routed in a channel located external to the pump head 88 such that the pump rollers or other pumping mechanism does not compress the return perfusate tubing 81. Once the pump 6 is activated, the pump head 88 will force the perfusate to flow from the perfusate source 4 into the source perfusate 80 tubing. The perfusate will continue to flow through the source tubing 80 and then transition into the first perfusate tube 48 within the junction point in the hub 82.

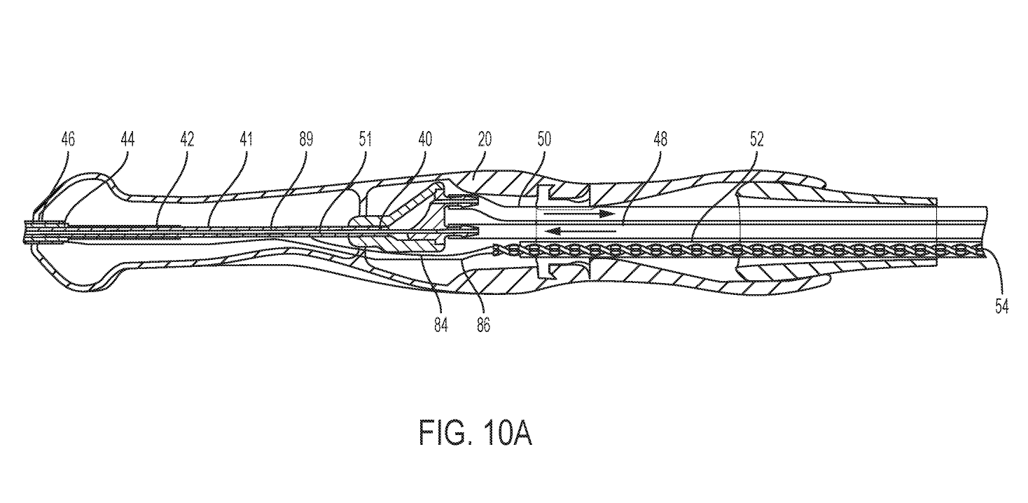

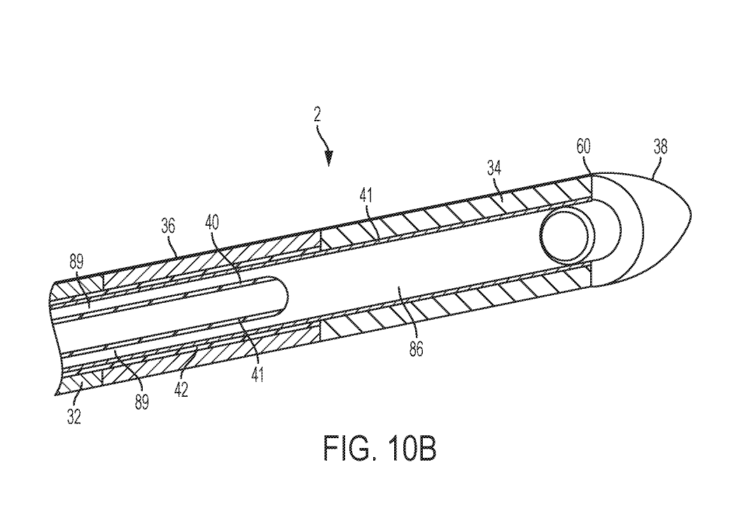

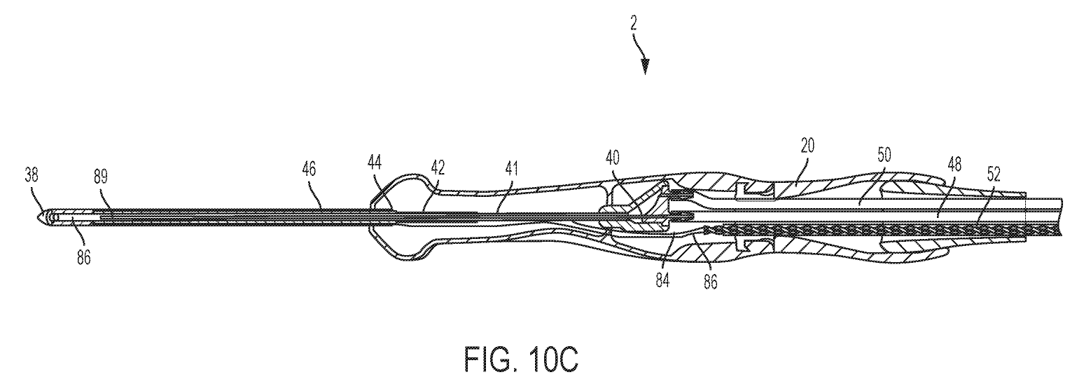

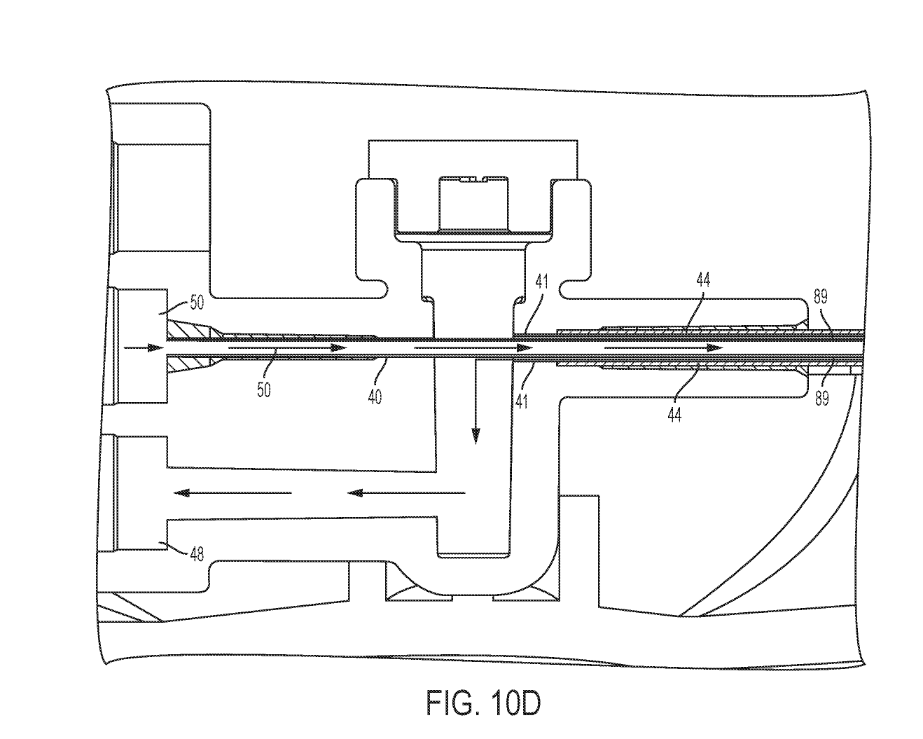

Referring specifically to FIG. 10A, in the handle 20 of the probe 2, the proximate end of the perfusate channel 40 is connected to and in fluid communication with the distal most end of the first perfusate tube 48. The perfusate will continue to flow through the first perfusate tube 48 and then enter into the lumen 51 of the perfusate channel 40; further illustrated by arrows in embodiment of FIG. 10D. The perfusate will then flow the entire length of the perfusate channel 40 and exit through the open distal end of the channel 40 entering into the lumen 86 of the first conductor tube 41, as shown in FIG. 10B. The distal most end 60 of the first conductor tube 41 is enclosed by the distal tip 38 as described above; such that the connection between the distal most end 60 of the first conductor tube 41 and the distal tip 38 prevents any perfusate from escaping outside of the probe 2 and into the tissue within the treatment zone. Since the pump 6 is continuously pumping perfusate into the system, this constant pumping force will circulate the perfusate contained within the lumen 86 of the first conductor tube 41 and force the perfusate to co-axially flow back up the first conductor tube through the coaxial return lumen 89 defined by the outer wall of the first channel 40 and the inner wall of the first conductor tube 41. This return lumen 89 extends the entire length of the probe shaft.

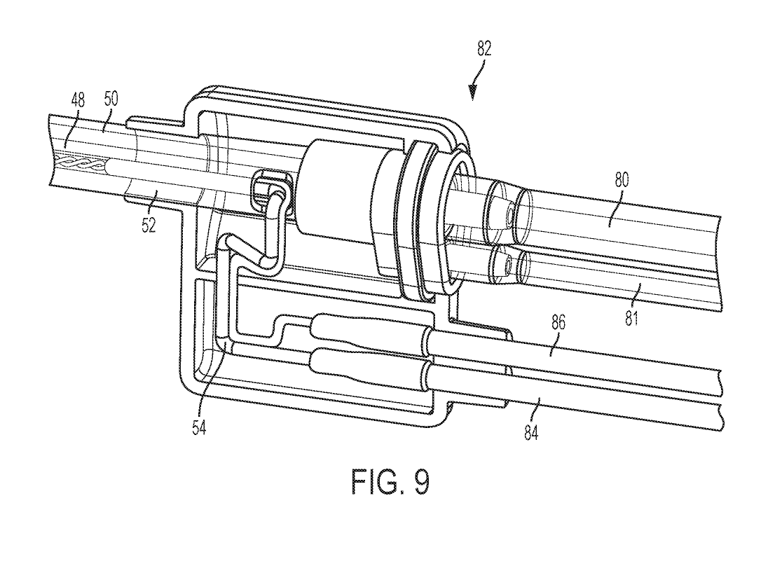

As shown in FIG. 10C-10D, the proximal most end of the first conductor tube 41 is in fluid communication with the second perfusate tube 50 within the handle 20 of the probe 2. As the perfusate is forced to flow into the return lumen 89 it will continue to flow along the length of the first conductor tube 41 in a distal to proximal direction into the second perfusate tube 50; further illustrated by arrows embodiment of FIG. 10D. The perfusate will continue to flow through the second perfusate tube 50 and then transition into the return perfusate tube 81 within the junction point in the hub 82, as shown in FIG. 9. The return perfusate tube 81 bypasses the pump head 88 and therefore the perfusate will be passively flowed into either a waste container (not shown) or back into the perfusate source 4 to be recirculated through the probe's fluid channels.

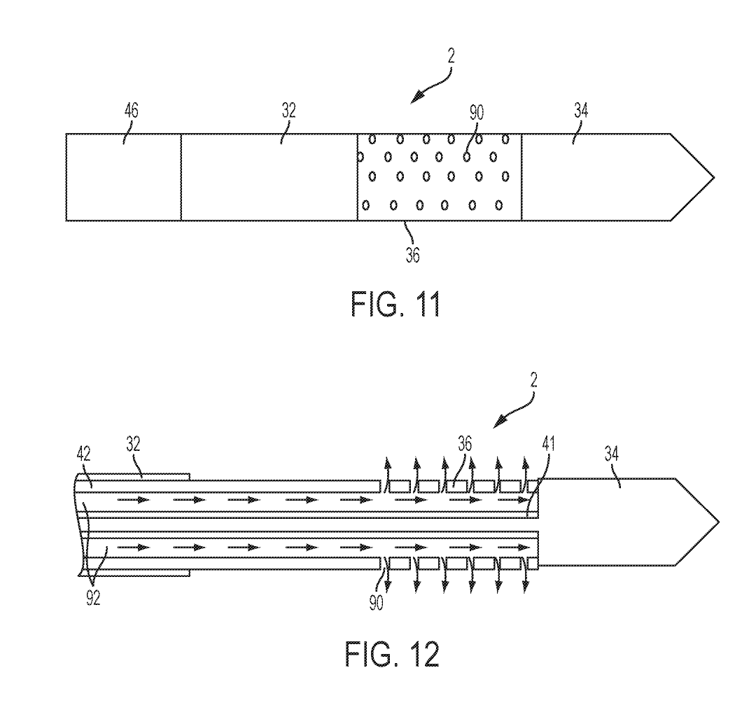

Referring now to FIGS. 11-12, other embodiments of the probe 2 are shown that include open or infusion designs. The purpose of the open perfusate embodiment is for the perfusate to pass through the probe 2 at a distal location and be infused or injected directly into the tissue. When the perfusate interacts directly with the tissue it may alter several aspects or physical properties of the tissue including, but not limiting to, the osmolarity, conductivity, and temperature of the interstitial space, as well as any secondary effects of targeted solutes within the perfusate such as drugs, immune antigens, or other cytoactive compounds.

Infusing perfusate through the probe and into the surrounding tissue may be one embodiment for solving the problem of arcing. Whereas a closed perfusion system solves the arcing problem by temperature control, the open perfusion system may solve the arcing problem by filling the air gaps created within the tissue during an IRE treatment with the perfusate, which is more conductive than air. It may be common for air bubbles and gaps to form during IRE procedures. The existence of air bubbles within the ablated tissues regions include, but are not limited to, the following: (a) the introduction of air when inserting the probe into the tissues or (b) the procedure itself, which can generate gasses as a result of the high voltages (electrolysis) produced during the procedures. For example, when electrical current passes through water, the H₂O molecule decomposes into O₂ and H₂ gasses. Electrolysis can occur in numerous other fluids as well. Air is generally highly resistive and could thus lead to arcing if it exists between the positive and negative electrodes during an IRE procedure that utilizes a monopolar probe or a bipolar probe. Filling these air gaps with a conductive substance, such as a perfusate, could potentially decrease the possibility of arcing during an IRE procedure by lowering the resistance of the tissue immediately adjacent to the electrodes.

As seen in FIG. 11, one embodiment of the open perfusion system includes the probe 2 having a series of infusion ports 90 along the spacer element 36 between the electrodes 32, 34. The infusion ports 90 may be holes, ports, pressure responsive slits, or other openings known in the art. The number of infusion ports 90 may vary depending on how much perfusate is desired to be injected or infused into the tissue. As seen in FIG. 12, the probe 2 of yet another embodiment for an open perfusion system has an infusion lumen 92 that run the length of the probe 2. In one embodiment, the infusion lumen 92 may be between the outer wall of the first conductor tube 41 and the inner wall of the first insulator tube 42. As the perfusate is pumped through the probe it travels down the infusion lumen 92 and will be infused or injected through the infusion ports 90 that are along the spacer element 36. In an alternative embodiment (not shown), there are no infusion ports along the spacer element. Rather, there is an infusion lumen between the inner wall of the second conductor tube 32 and the outer wall of the first insulator tube 42, and there is an infusion gap (not shown) between the distal most ends of the second conductor tube 32 and the first insulator tube 41. The perfusate of this embodiment would flow through the infusion gap and exit the probe through the infusion gap, thereby infusing or injecting perfusate into the tissue within the treatment zone.

In yet another embodiment, the infusion ports 90 are located at the edges of the spacer adjacent to the closest edges of the electrodes where arcing is most likely to occur.

The delivery or circulation of perfusate may be controlled by a combination of the pump 6 and control unit 12. The user may be able to input 16 various parameters of the perfusion through the graphical user interface (hereafter “GUI”), which is viewable on the display 10, and in turn is controlled by the control unit 12. The control unit 12 may be programmed to automatically adjust various parameters or settings of the pump 6 based on user inputted parameter thresholds, which in turn will control the flow, or lack thereof, of the perfusate. It is within the conception of this invention that various parameters of the perfusate may be controlled, changed, altered, or otherwise affected. Such parameters of the perfusate may include, but are not limited to, the following: the matter state (gas/liquid); the electrical conductivity; osmolality or concentration of the perfusate; thermal conductivity; heat capacity; temperature of perfusate; flow rate of perfusate through system; delivery of perfusate during only certain pulse sets/trains; and timing of the perfusate (before, after, or during an IRE pulse delivery or treatment), thereby maximizing ablation zone size and mitigate late-onset thermal damage.

The system may allow the user to select various options for when to deliver perfusate. For example, the user may be able to input or select on the GUI from various “perfusate delivery” options. Such options, which are described below in more detail, may be preset options on the system or may be added/customized by the user. When the user selects a perfusate option the control unit then triggers the pump to deliver perfusate based on user requested settings, such as at a particular flow rate, at a particular time during the procedure, or at a certain temperature threshold.

In one embodiment, the control unit 12 triggers the pump 6 to deliver perfusate only during delivery of the IRE pulses, but does not perfuse during inactive states of the pulse protocol. In this embodiment, the control unit 12 may trigger the pump 6 to flow perfusate when the electrode(s) are in an active sequence state, but not during deliberate pauses in phased pulse delivery or not during delays between pulse trains, such as during the 3.5 s delay between trains of 10 pulses as will be described in more detail below.

In yet another embodiment, the control unit 12 triggers the pump 6 to deliver perfusate only when the temperature of the electrode has reached a threshold. The user may be able to select on the GUI either an upper threshold or lower threshold of temperature. The upper threshold may be the temperature setting above which thermal damage to the tissue may occur or has been deemed to cause increases in electric conductivity and electric current that risk exceeding the electric current specifications of the electroporation pulse generator, or risk inducing an arc. The lower threshold may be the temperature setting below which insufficient ablation volumes are produced; where the temperature being too low risks adversely affecting the ablation zone due to redistribution of conductivity and electric field; or where the lower temperature risks instability in the pulse. For example, too sharp of a contrast between the cooled electrode and warmed tissue may cause irregular electrical current behavior and increased likelihood of arcing.

In yet another embodiment, the control unit 12 triggers the pump 6 to deliver perfusate only when the electrical current of the pulse has reached a predetermined threshold. For example, perfusate may be delivered only to reduce the tissue and electrode conductivity in order to reduce electrical current. Moreover, the predetermined threshold may be an absolute level which is detected or otherwise sensed by the system that suggests current will soon exceed specifications of the electroporation generator, or suggests that there is considerable risk for electrical arcing. As a non-limiting example, if the system detects that arcing only occur once the current exceeds 35 A, the current threshold can be set at or just below this amperage value. Once the system detects the current threshold has been reached, the perfusate flow is automatically triggered or modified to maintain the detected current below the preset critical threshold. The predetermined threshold may be a relative value based on a pre-pulse low-voltage electrical current, or the predetermined threshold may be a relative value based on initial electric currents for therapeutic pulses.

In yet another embodiment, the control unit 12 triggers the pump 6 to deliver perfusate only when the electrical current displays an unstable waveform suggestive of an impending arc due to oscillations of electric current, at least two- or multi-plateau waveforms, or a sudden rise in current within but at the end duration of a pulse.

Another key aspect of this invention is the ability to control the temperature of the perfusate for either an open system or a closed system. In one embodiment, the system of this invention is able to control the temperature of the perfusate as it relates to the impact that such a temperature change would have on the delivered pulse parameters, and in turn the effects of the delivered IRE pulses on the tissue. It has been found that room temperature and body temperature perfusate result in clinically acceptable ablation sizes and use less power than chilled perfusate, which may result in a lower likelihood of potential arcing.

The temperature of the temperature control perfusate can be active or passive. Passive temperature controlled perfusion is when a large amount of the perfusate at a set temperature is stored in a reservoir 4, such that any perfusate coming back into the reservoir 4 will not significantly change the temperature inside the reservoir 4. The control unit 12 will monitor the reservoir 4 temperature and warn the user if the temperature rises above the threshold high temperature. Alternatively, the control unit may automatically adjust temperature levels based on the user-defined threshold. Active temperature controlled perfusion is when the perfusate temperature is monitored by the control unit 12 using a temperature sensor (not shown) on the probe inside the tissue and near the electrode, and if it rises or falls below the set temperature levels, the control unit 12 can activate a temperature control device associated with the perfusate reservoir to automatically adjust the temperature of the perfusate.

Examples of controlling the temperature of the perfusate may include, but are not limited to, (i) changing the temperature of the perfusate relative to ambient body temperature throughout the procedure; (ii) dynamically changing the perfusate temperature during the procedure, such as starting with a lower temperature perfusate and ending the procedure with a higher temperature perfusate or starting with higher temperature perfusate and ending with a lower temperature; (iii) independently controlling temperature of perfusate for each electrode and/or for each probe if multiple monopolar probes are being used; (iv) setting the temperature of perfusate based on the tissue type being treated; and (v) controlling the temperature of perfusate based on comparison of the real-time temperature of the electrodes to a pre-set temperature threshold, e.g., utilizing an algorithm to initiate cooling perfusate when/if the electrodes reach are preset temperature, such as 45° C. The temperature of the perfusate may be either chilled (about 10° C.); ambient or room temperature (about 20° C.); or body temperature (about 37° C.).

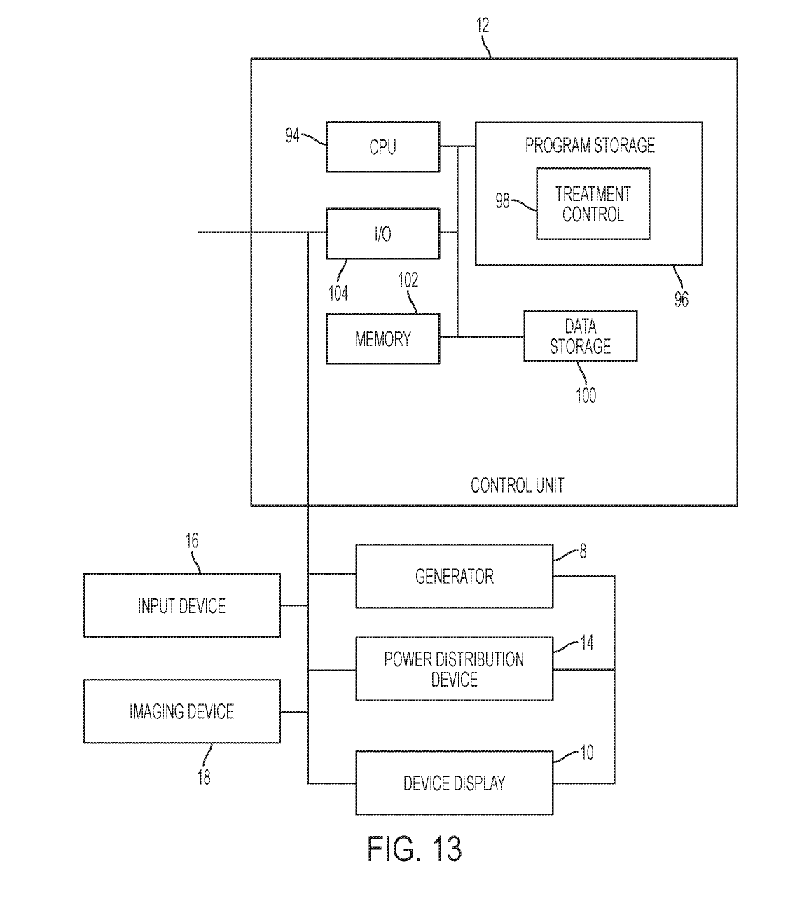

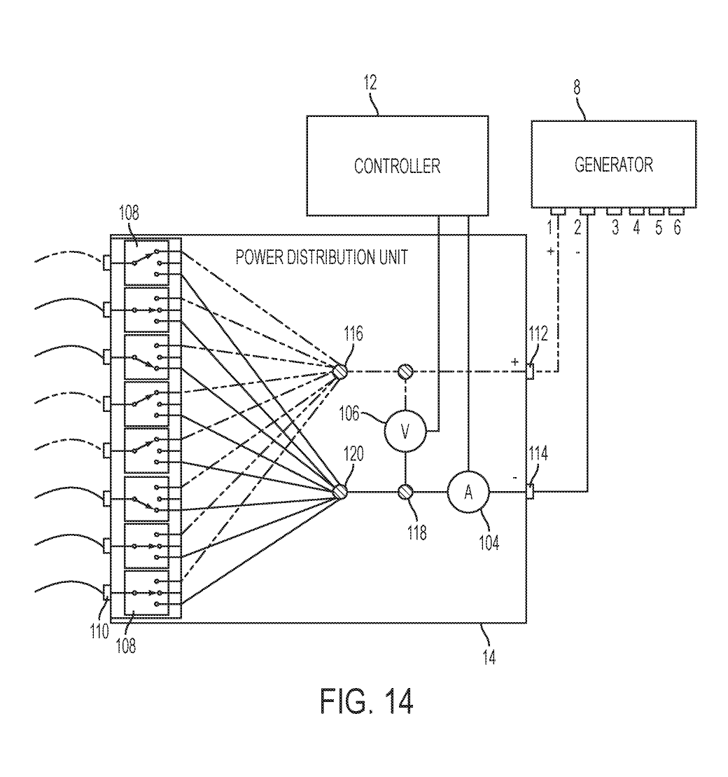

Referring now to FIGS. 13-14, another key aspect of this invention is the control unit 12 that controls the pulse parameter settings, the generator 8 used to deliver the electric pulses, and the integration of the other system components used to generate, control, display, deliver, and monitor the electrical pulses. While current commercially available EBT pulse generators 8 are restricted to ˜3400 V, they are also unable to manage more than 50 Amperes of electrical current during each pulse. In addition, typical electrode geometries and pulse delivery protocols often result in physical conditions where larger voltages will result in arcing in the tissue. As previously discussed, arcing can cause problems with operation of the generator 8 and impede the successful delivery of energy into the tissue. Arcing problems may cause an automatic shutdown of the generator 8, aborted treatment procedure, and/and increased procedure time needed to reboot the generator 8 after a shutdown. The improved generator 8 of this system solves these and other problems known in the art.

The controller 12 may be able to provide the user with real-time feedback and pulse parameter control. The controller may include computer program storage or software 96, which further comprises various treatment control options 98, data storage 100, a CPU 94, power source 104 and memory 102. The controller 12 is designed to assist a user in planning, executing, monitoring, storing, retrieving, and reviewing the results of an IRE medical treatment procedure. For example, in one embodiment of this system the controller 12 provides a GUI interface on display 10 which allows the user to select various treatment control 98 options, such as the tissue type to be treated and/or the size of desired ablation zone. Other treatment control 98 options that the GUI provides may include customization of various pulse parameters of the pulses to be delivered, including, but not limited to, the pulse length, number of pulses per train, number of pulse trains, length between each pulse, length between each train, or the overall length of pulse delivery. The controller 12 may also be connected to a power source 104 or have an internal power source (such as a battery). Moreover, additional software 96 may be stored in the data storage component of the controller 12 to provide the user with 3D reconstructions of the treatment site and with overlay of predictive ablation zones on the display such that the user to be able to better formulate and execute a treatment plan.

In another aspect of the improved generator 8 of this invention, the generator includes an automatic recharge feature in which the system recharges after each pulse is delivered. This design eliminates the voltage decay which occurs over a pulse train seen in current generators and provides a more consistent voltage delivery that better matches the user input. The generator 8 may also be capable of generating or alternating polarity between pulses (bipolar pulses) and/or between pulse trains by using additional capacitors. Another advantage of the generator 8 may be to eliminate a hard shutdown after an arc has occurred, thereby allowing IRE procedures to progress without system data loss. It may also be conceivable to allow the user to view real-time pulse metric data, such as voltage, current, and/or resistance.

As described above, an advantage of this system is to provide a compact device with a small footprint. In addition to integrating the pump 6 within the generator 8, as described above, it may be possible to also integrate an ECG synchronization device (not shown), which are commonly known and used in current IRE procedures, into the generator 8 housing.

The generator 8 may be designed to support up to support up to 12 probes, either monopolar or bipolar probes with optional RFID readable technology integrated therein. The RFID technology may be used for detecting the probe type, identifying probe configuration, confirming single use of each probe, confirming of correct connection, and prohibiting the use of probes not compatible or not intended to be used with the generator.

The generator 8 may also integrate a means to measure the endpoint of a procedure. There is a current need in the art for the pulse metrics, such as voltage, current, and resistance, to serve as valuable indicators to the progression of IRE; both from the intra-pulse IRE data as well as the inherent tissue properties after exposure to a set of IRE pulses. For example, a need in the art currently exists for a clinically acceptable IRE system that can indicate potential issues that may require intervention during an IRE treatment to prevent superseding the capacity of the generator and arcing, as well as to display or notify the user of the extent and thoroughness of electroporation, particularly in the ablation zone. While current commercially available IRE systems do not provide these pulse data in real-time, the improved system of this invention solves the need of deriving this information from the pulses in real-time during treatment delivery. By doing so, those skilled in the art of delivering IRE treatments can prevent potentially problematic conditions, make necessary adjustments, preventing issues that may diminish optimum therapy delivery. One purpose of this invention is to provide the user with the ability to determine if either the IRE pulse strength is too low or too high, if the system is at risk of arcing, and as well as indicating when the tissue between a given pair of electrodes has been completely electroporated or treatment is complete. Since the current IRE treatment devices do not provide the user with the ability to be notified or even visualize when the endpoint of a procedure has been successfully reached, one embodiment would be to use a low voltage measurement system integrated into the generator 8 to monitor for procedure endpoint. For example, the generator 8 could be built with two capacitor banks or two circuits, one for high voltage and one for low voltage. Low voltage measurements may be separately monitored during treatment to detect conductivity changes in the tissue as a result of high voltage pulse delivery. Alternatively, another embodiment could be an AC spectroscopy or an AC frequency sweep to compile real-time conductivity changes in the targeted tissue.

The system display 10 may be a standard display 10 that is currently known in the art. The display 10 may also be a tablet, smart phone or other portable computer capable of wirelessly connecting to the controller over Wi-Fi or other wireless means. The display 10 may also be used together with an imaging device 18, such as an ultrasound device, to provide the user with the ability to scale the image so they can place the tumor in context of the patient's body. Additionally, the system may be comprised of multiple screens or multiple displays 10. For example, a first screen may display 10 the current, resistance, or treatment parameters, and a second screen may have a user GUI and/or ultrasound/MRI/CT image.

As part of the pre-planning process, the system may also be able to import CT scans or other imaging device output ages and overlay these images with the modeled electric fields (current, electrical field distribution) used to predict tumor volume.

The system may also include a power distribution unit 14 to control the energy delivery of IRE electric pulses. The power distribution unit 14 is intended to solve the need in the art for an IRE system with an enhanced power distribution of the electric pulses and provide necessary real-time electrical pulse data. This may include being able to use more than the six controllable electric pulse outputs of current IRE systems. Additionally, there is also a need for an IRE system with the ability to alternate the polarity of the pukes at determined intervals or in response to therapy behavior, such as arcing occurring with pulse delivery, which can at times be eliminated by alternating the polarity; reduce total charge delivered; reduce electrochemical effects of pH imbalance; and mitigate discrete gas element formation from electrolysis. Moreover, current state of the art IRE generators are only able to energize two electrodes at a time, with one serving as the anode and the other as the cathode. If more than two electrodes are required for a treatment, commercial generators currently will sequentially alter what the two energized electrodes are, requiring exponentially higher numbers of total electric pulses as the number of electrodes and thus combinations of electrode pairs increase. This may create problems with requiring significant time increases to treat larger tumors that require more electrodes; which at times can restrict the practical utility of IRE therapy or result in incomplete treatment due to the time constraints for delivering therapy to an anesthetized patient. In addition, there are many instances where more effective IRE therapy delivery may require energizing several or many electrodes simultaneously, as explained in more detail below. The present invention enables activating any number of electrodes to serve as either the positive or negative portion of the pulse pair.

Current commercial systems for clinical applications of IRE pulses are only display electric pulse metrics after completion of an entire procedure, and are limited to delivering electric pulses through a total of six outputs or probes. However, there are many clinical cases that may require greater than six outputs or probes to serve as anodes or cathodes throughout the duration of a procedure, due to either large ablation volume requirements or complex tumor geometries. This includes the utilization of greater than six, single pole electrodes used in an array, as well as specialized electrodes which may contain a large number of individual contact surfaces for the delivery of IRE electric pulses. This system solves these unmet needs in the art through the use of a power distribution unit 14. As seen in FIG. 14, the power distribution unit 14 may be connected to the generator 8 and/or control unit 12 with standard connections known in the art, which in turn is connected to the display 10 as shown in FIG. 13, such that the system is capable of monitoring and displaying the electric pulse metric during delivery in real-time, which may offer beneficial information and feedback to improve clinical applications of IRE protocols. In another embodiment, the power distribution unit 14 may be incorporated into the same housing as the generator 8 to achieve a smaller overall system footprint.

In one embodiment, the power distribution unit 14 may consist of an ammeter 104, a high-voltage voltmeter 106, an array of switches 108 that have three positions (off, on-positive, on-negative), and a series of probe outputs 110. The power distribution unit 14 may receive the electric pulse signal from the generator 8 through designated positive input 112 and negative input 114. The positive input 112 is connected to a positive distribution node 116 and the positive terminal of the voltmeter 106. The negative input 114 is connected through ammeter 104 to a negative terminal 118, which is connected to a negative distribution node 120 and the negative terminal of the voltmeter 106. The positive distribution node 116 is then connected to the positive terminal of each switch 108. The negative distribution node 120 is then connected to the negative terminal of each switch 108.

In one embodiment, the generator 8 may be set to deliver energy to each of the outputs in the same manner. For example, a first output may always be positive and connect to the positive input 112 of the power distribution unit 14, while a second output may always be negative and connect to the negative input 114 of the power distribution unit 14. When an electric pulse is delivered, the electrical energy goes through the power distribution unit 14, and any switches 108 set to positive positively energizing any probe connected to that probe outputs. The electrodes connected to the negative pole of the switch 108 then return how much voltage is left after the pulses have been delivered to the tissue back to the power distribution unit 14. The negative signal then returns to the negative distribution node 120, runs through the ammeter 104, and then returns back to the generator 8. The voltmeter 106 measures the voltage drop between the positive and negative signals to measure the total voltage delivered to the tissue. The outputs from the voltmeter 106 and ammeter 104 are then sent to the controller 12 and/or display 10 so both the voltage and current pulse metrics delivered to the tissue can be visualized in real-time. The controller 12 may include software 96 to compute calculations of these signals so they can be used to determine the tissue resistance or conductance, as well as numerous other parameters resulting from the electric pulse metrics.

In another embodiment, the power distribution unit 14 may also include components and circuitry (not shown) to measure real-time feedback parameters, including, but not limited to, resistance; impedance, frequency, and specific impedance. These measured parameters may then be depicted on the display 10 to allow the user to integrate this information into the treatment planning. In addition, in yet another embodiment, the power distribution unit 14 may receive pulse energy from at least two inputs, such as a positive and a ground, and facilitates individual control of how the electrical voltage from the generator is distributed among electrodes. The invention is therefore unlimited in the number of electrodes that in can distribute the power to over the course of a procedure. Furthermore, the power distribution unit 14 together with the controller 12 and generator 8 may also enable complex distribution patterns and algorithms to fine tune ablation volumes/geometries of multiple electrodes serving as a positive or ground simultaneously. This may have benefits for shaping the electric field distribution, as well as decreasing the overall IRE procedure time by permitting pulse delivery between several electrodes simultaneously, rather than serially, when the total distributed electric current is maintained within the constraints of the generator 8. This decrease in overall procedure time would be a significant advantage over the currently commercially available IRE treatment systems.

In another embodiment, the power distribution unit 14 may also include a metric measuring functionality. For example, the ammeter 104 may be replaced with a Hall Effect probe (not shown), so as to measure the electric current without directly interfering with the pulse signal. Alternatively, a unique ammeter may be placed on the negative signal connection between the switch and the node for each individual switch, so as to measure the electric current of each negative electrode separately. Moreover, the high-voltage voltmeter 106 may be replaced with a basic voltmeter placed on a voltage divider circuit. The voltage divider circuit is of much higher resistance than the tissue (kΩ-MΩ, versus tissue, which is hundreds of Ω), and thus inducing negligible effect on the strength of the pulses delivered to the tissue. A correction factor is calculated by the controller for the divider circuit based on the resistances of the three resistors in the voltage divider circuit to determine the true delivered voltage to the tissue based on the measured voltage drop across the resistor. An accurate voltage is required to attain accurate resistance measures.

Additional embodiments of the power distribution unit 14 may include a double pole double throw switch placed between the distribution nodes 116, 120, but internal to the voltmeter and ammeter sections. This switch is connected to the positive and negative portions on both ends of the switch, but with connections on opposite ends leading to the distribution nodes 116, 120. This enables the switch to function as a rapid switch to reverse the polarity being sent to all active electrodes at once. A fuse may be integrated into circuit on the positive end just after the input of the power distribution unit 114. This fuse is fast-acting, and is able to trigger in the instance of a high current condition. This would enable the fuse to trigger and cease energy delivery to the tissue prior to any arc issues being encountered by generator 8. The power distribution unit 14 may include an oscilloscope to monitor pulse metrics in real-time.

Additionally, a switch may be introduced to the positive side of the signal between the voltmeter and positive distribution node 116. The switch is a single pole double throw switch. When the switch is in one position, thy: signal continues to the positive distribution node 116. When the switch is in a second position, the system moves through a relatively high impedance resistor, effectively dropping the voltage delivered to the tissue. This enables delivery of lower voltages to the tissue than current commercially available electroporation generator maybe capable of delivering. Additionally, this may also allow for rapid high voltage and low voltage pulse delivery without charging the capacitor bank, eliminating the need for a second capacitor bank or charging/discharging delays. The use of low voltage pulses includes determination of baseline tissue properties and tissue properties in the absence of any engaged electroporation phenomenon. An additional resistor may be added and an adjustable potentiometer, enabling control to tune exactly how much the voltage will be dropped before reaching the positive distribution node 116 and electrodes. The voltmeter 106 may be placed after the switch to enable measuring what the effective voltage delivered to the tissue. Adjustable resistance potentiometers may be placed on the positive connection to each switch individually. Such an arrangement would enable the user to fine tune which electrode connections receive larger/smaller voltages based on the electrode geometry and tissue conditions of the targeted region. Some separations may be larger d require larger voltages than closer electrodes, among many other possibilities where this would be useful. Lastly, the power distribution unit may also be integrated directly into the generator, before the generator's 8 conventional outputs. In this way, the switching system is controlled from the generator 8.

The method of using this system to treat issue will now be described. The method may include reducing the temperature of the affected tissues as a way to reduce local tissue conductivity, both over baseline in general, as well as relative to what conductivities would be encountered for higher temperature tissues. One key inventive concept of this system and the method of using the system is controlling the temperature rise of the treatment zone and its correlated electric conductivity, thereby making it possible to deliver higher voltages reliably into tissue without the risk of arcing or exceeding 50 A (which is the current limits on commercially available IRE generators), thus enabling greater electric field distributions and affected volumes.

In one preferred embodiment, the method of using the single stick bipolar system probe may include perfusion of internally circulated room temperature perfusate within the probe together with the pulse parameters of five pulses per train; an inter-cycle delay of two second between pulses, ranging between 0.5 and 3 seconds depending on the patient's heart beat; a set of two trains per row; and a ten second delay between each row. This combination of perfusion together with the specific pulse parameter set has been found to achieve clinically acceptable ablation zones using a single stick bipolar probe while reducing the potential for unstable electric current and/or arcing potential between electrodes.

In a situation where the temperature of the electrode and/or tissue is controlled via perfusion, either directly infusing perfusate into the treatment site or indirectly by internally circulating perfusate within the probe, the dependence of electrical conductivity on temperature is altered, thus affecting the electric conductivity of the tissue relative to its conventional behavior in response to the Joule heating from the IRE pulses. It is possible that the high number of total pulses delivered for a commercially available IRE device may result in significant cumulative thermal effects, with temperatures shown to reach levels as high as 59° C. in regions between several pulse pairings at typical pulse protocols. It has been found that temperature rise may alter the properties of the tissue within the treatment zone and thus alter treatment procedure outcomes. For example, when the temperature rises during an IRE procedure are not evenly distributed this may result in heterogeneous conductivity distribution which may significantly alter the electric field distribution in the tissue within the treatment site. Using perfusion the method of use may include reducing the resistance of the hottest regions of the tissue or the probe 2, which are typically adjacent to the point at which the electrodes 32, 34 are closest together, reducing the bulk tissue conductance, and thereby reducing the overall electric current delivered for a given electroporation pulse voltage. Perfusion makes the electrical field distribution more homogeneous by leveling balancing tissue conductivity within the target area. Advantageously, reducing the overall electric current delivered for a given electroporation pulse voltage may enable greater voltages to be delivered to the treatment zone, thus increasing the ablation volume of IRE treatment using just a single stick bipolar probe.

While unstable electric current and arcing between electrodes are often correlated they are in fact two different modes by which an IRE generator or system can fail during a treatment leading to an overall reduction in voltage delivered to the tissue, thereby resulting in an incomplete or failed treatment procedure. Perfusion of the electrodes may address unstable electric current and arcing between electrodes, thus allowing for an overall increase in the voltages that can be delivered to the treatment zone. It has been discovered and described in more detail below, that perfusion of a bipolar probe using ambient or room temperature perfusate may increase general pulse electric current stability and reduce sudden spikes in current, thereby reducing the potential for arcing between electrodes. Moreover, it has been found that perfusion via internally circulating room temperature perfusate within the bipolar probe reduces temperature at the hottest portions of each electrode and adjacent the tissue within the treatment zone resulting in a reduction of sudden inflections and arcing between electrodes. Therefore, perfusion of a single stick bipolar probe may exhibit a reduction in the bulk tissue temperature rises and maximum temperatures, dramatically reducing the extent of thermal damage including the variety of thermal damage that risks morbidity to the sensitive structures within the treatment zone while still achieving clinically significant irreversible electroporation of cells.

This system may provide for the ability to control and alter the temperature of the perfusate in real-time during the procedure, or to use a room temperature perfusate that does not require any active temperature control. Multiple studies were performed using this system to determine the effect of different temperature perfusate and the impact it had on achieving the desired goal of enabling larger electric field distribution size and affected volumes. As will be described below, the method of using this system was tested with perfusion that was continuously cooled or chilled, at ambient room temperature, and also maintained at body temperature.

In a first experimental study, a perfused bipolar electrode, similar to the probe described above, of dimension 10 mm×7 mm×10 mm (energized, insulating separation, energized) as inserted into either room temperature potato or liver. Three temperature solutions were perfused to determine possible effects of perfusion: chilled perfusate (8° C.), room temperature perfusate (21° C.), and body temperature perfusate (37° C.). The three temperature-controlled and perfused probes were compared with a non-perfused probe, such as is commercially available. The perfusate was 0.9% NaCl solution was used as the perfusate. A maximum perfusion setting was used on a peristaltic pump to deliver perfusate to the probe during pulse delivery. Voltages applied to the electrodes were varied to determine the maximum permissible voltage for each perfusate without arcing.

The temperature-controlled probes showed maximum temperatures equivalent to their respective perfusate temperature in both potato and liver, suggesting the perfusion rate and perfusate fluid was sufficient to maintain the electrode at the targeted temperature throughout the duration of the procedure. In potato, the maximum temperature reached by the ambient, meaning no perfusate was used, probe with no perfusion was 62° C., despite it starting with an initial temperature of room temperature (rise of approximately 40° C.). In liver, the maximum temperature reached from the ambient electrode was 65° C., giving an increase of ˜35° C. above its baseline of the tissue.

In potato, 2000 V was applied. The body temperature perfused probe and non-perfused probe showed equivalent trends of rise in electric current, while room temperature probe and chilled probe showed reductions in electrical current of 10% and 20%, respectively. In liver, all three temperature controlled and perfused probes showed less electrical current delivered during the procedure compared to the non-perfusate probe, with maximum reductions in current of 47% for body temperature perfused to probe, 52% for room temperature probe, and 65% for chilled temperature perfused compared to the non-perfused probe.

All three temperatures controlled and perfused probes showed a reduced likelihood for electrical arcing compared to the non-perfused probe. In potato, average number of arcing events over the entire pulse duration was <1 for all three temperature controlled and cooled probes; while it was 3.75 for the non-perfused probe. In liver, arcs were only noted in non-perfused probe (3.5 average) and chilled perfusate probe (1.5 average), suggesting that body temperature and/or room temperature perfusate may be more stable at preventing arcs in the tissue even if their electric current is found to be higher.

In potato, there appeared to be no significant correlation between ablation zone size and perfusate temperature, or relative to the non-perfused probe. This would suggest that the changes in perfusion may be used without an effect on ablation zone. As general trends, the non-perfused electrode performed the worst (smallest lesion) while the body temperature perfused probe showed the largest ablation area.

This first experimental study found all three temperature controlled perfused probe significantly reduced the number of arcs and maximum temperatures reached in the tissues compared with a non-perfused probe. Below is a table summarizing the results of the first experimental study.

TABLE 1
Comparing Temperature Controlled Perfusion probe with Non-Perfused
probe.

| | Max. | | |
|---|---|---|---|
| | Temp | % of Current Reduction | Ave. number |
| | (Electrode | (Liver) relative to Non- | of Arcs |
| Type of Perfusate | Surface) | perfused | (Liver) |
| None | 65° C. | — | 3.5 |
| (Non-perfused) | | | |
| Chilled | 8° C. | 65% | 1.5 |
| 8° C. | | | |
| Room Temperature | 21° C. | 52% | — |
| 21° C. | | | |
| Body Temperature | 37° C. | 47% | — |
| 37° C. | | | |

In a second experimental study, the effects of chilled perfusate that having a temperature of approximately 10° C. was further compared with a system that has no perfusate. Voltages applied to the electrodes were varied to determine the maximum permissible voltage for perfusate vs. non-perfusate. It should be noted that all simulations use dynamic conductivity in the sense that conduct is changing as a result of rising temperature. However, the terminology used here will delineate between static and dynamic conductivity simulations based on whether electroporation-based conductivity rise is included in the model, while temperature is always considered. The results of this study are shown in Table 2 below.

TABLE 2
Chilled Perfusate vs. No Perfusate.

| Conduc- | | | | Overall | Overall |
|---|---|---|---|---|---|
| tivity | | Electrical | Total Volume | Volume | Volume |
| Condition | Cooling | Current | E >500 V/cm | T >50° C. | T >70° C. |
| Static | None | 44.68 A | 10.64 cm³ | 6.64 cm³ | 2.66 cm³ |
| Static | 10° C. | 14.07 A | 5.15 cm³ | 0 cm³ | 0 cm³ |
| Dynamic | None | 89.1 A | 14.8 cm³ | 8.01 cm³ | 3.80 cm³ |
| Dynamic | 10° C. | 23.9 A | 8.04 cm³ | 0 cm³ | 0 cm³ |

Based on the data in Table 2, the test data clearly showed that the non-perfused electrode causes very high temperature changes in the tissue, with volumes of 6.64 cm³ and 2.66 cm³ reaching temperatures above 50° C. and 70° C., respectively, for the static, as well as 8.01 cm³ and 3.80 cm³ for the dynamic conductivity model. However, there is much less temperature rise for the perfused probes using a chilled perfusate, with no volume of the tissue reaching temperatures above these thresholds at all. This suggests that the chilled perfusate would render minimal or no thermal damage. The use of chilled perfusate was shown to reduce the electrical current by 69% and 73% for static and dynamic conductivity simulations, respectively. This indicates that significantly higher voltage electric pulses can be used while remaining below the 50 A threshold for commercially available generators when chilled perfusate is incorporated into the system.

Although chilled perfusate reduces the electric current and temperatures that the tissue is exposed to, it also affects the conductivity and thus electric field distribution through the tissue. For the conditions examined, the predicted ablation zone decreases by 52% and 46% for static and dynamic simulation conditions, respectively; with the predominant effect occurring in the ablation zone diameter rather than its length.

The use of chilled perfusate together with the above described system may likely allow higher voltages to be used by reducing the electric current and extent of unintended thermal damage to tissue immediately surrounding the electrodes. However, for the same voltage, the ablation zone may be smaller and less spherical (for certain electrode embodiments). Thus, it is desirable for the present invention to optimize the balance between benefits on electrical behavior, while minimizing the extent of reduced ablation volume and minimizing the possibility of arcing. If this balance can be achieved, the use of perfusate with the present system should enable larger ablations to be achieved, with more consistent and reliable energy delivery that may be more readily applied repeatedly across numerous clinical scenarios.

In a third experimental study, the use of body temperature perfusate was further examined. A perfused bipolar probe, similar to the probe described above, with dimensions of 10×7×10 mm (energized, insulating separation, energized) was used. A total of 19 ablations in muscle and liver were done to determine the effect of varying active cooling algorithms on procedure outcomes. During this in vivo test, body temperature perfusate resulted in clinically acceptable ablation size and used less power than chilled perfusate. The use of body temperature perfusate (either during delivery or turned on when T>50° C.) resulted in the largest ablation zone diameter, with 3.2 cm relative to 3.0 cm for room temperature perfusate and 3.1 cm for continuously cooled perfusate. In addition, the average maximum electrical current for triggered body temperature perfusate was 24 A, which is lower than the 33 A and 26 A for room temperature and chilled electrodes, respectively. Moreover, the average incidence of arcs found for the triggered body temperature perfusate was 2.8 compared to 6.5 for the room temperature perfusate and 4.8 for chilled perfusate. This data suggests that continuous circulation of body temperature perfusate and ideally triggered body temperature perfusate may result in greater reliability for being able to deliver an entire ablation protocol without exceeding electroporation generator electric current limits and also with fewer incidence of arcing compared with non-perfused systems. The inclusion of the body temperature perfusate markedly decreased the volume and extent of thermal damage to the tissue, thus better preserving IREs unique non-thermal cell death modality.

Continuous delivery of body temperature perfusate may provide a less intense cooling regimen to attain equivalent benefits on pulse stability and reduced electrical current, while providing more reliable energy delivery and higher applied voltages, but ideally without as significant of a redistribution in electric conductivity and electric field, offering larger ablation zones. The use of body temperature perfusate may result in less arcing and greater pulse stability than when using chilled perfusate, despite the additional cooling of the chilled perfusate.

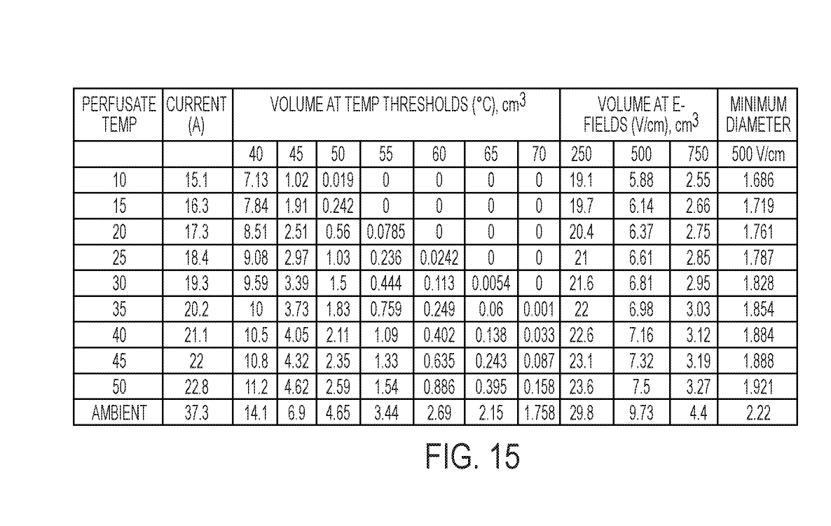

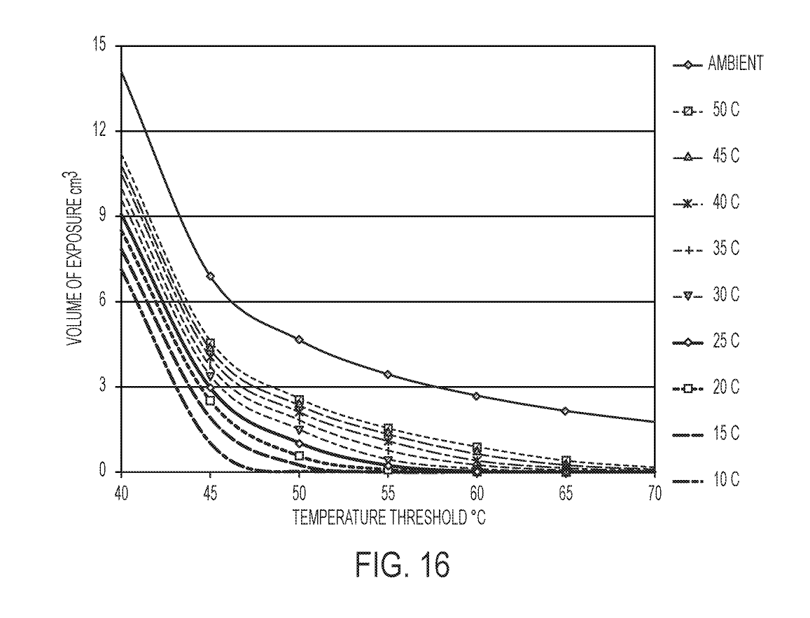

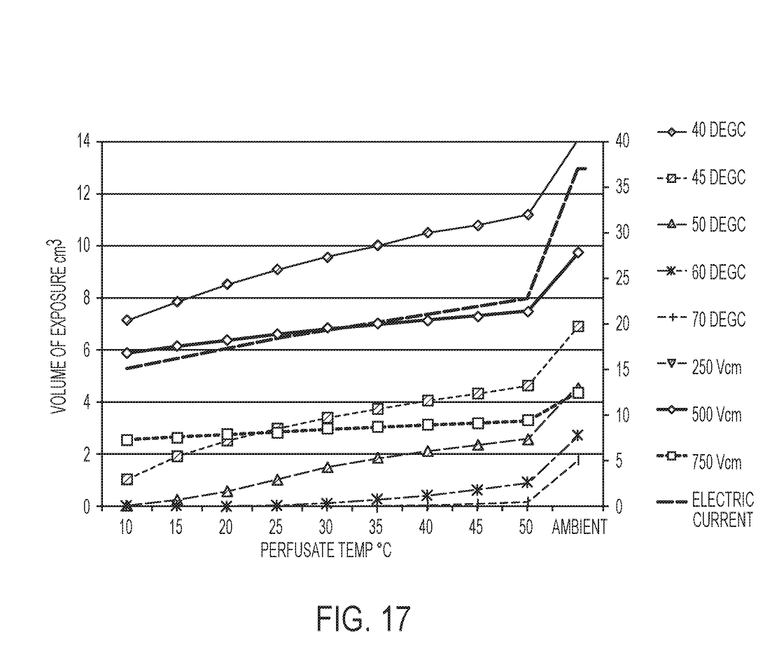

A simulation, created with Comsol Multiphysics v3.5, was conducted to support the findings from the benchtop and in vivo animal studies, i.e., a marked increase in ablation zone when using warmed perfusate or a room temperature perfusate relative to chilled perfusate. The results of this simulation are shown in FIGS. 15-17. Importantly, the use chilled temperature perfusate, room temperature perfusate and body temperature perfusate all showed notable decreases in electric currents required to achieve clinically acceptable ablation volumes, thermally suspicious areas, and arc incidences compared to non-perfused systems. The objective of this simulation was to analyze perfusion with varying perfusate temperatures in order to determine a crossover point/range where benefits to reduced currents and thermal damage are maximized while volume of affected region exposure is maintained at a clinically level. It was discovered that the non-perfused or ambient probes had much greater thermal exposure volumes compared to all of the temperature controlled perfusate, with progressively lower exposure volumes as perfusate temperature decreased. Thermal exposure volume is defined as that portion of the total ablated tissue volume subjected to temperatures of 50° C. or greater. Temperatures reached 70° C., which is relevant for damage to collagen, in greater than 0.1 cm³ of tissue perfused at 50° C. or ambient conditions. Volume of thermal exposure to 70° C. was less than 0.1 cm³ for all other perfusate temperatures, showing they should all be negligible in creating thermal tissue damage. Even at 50° C. perfusion, the exposure volume is only 0.158 cm³, which is still less than 1/10th that of the ambient probe's 1.76 cm³ measurement, as seen in FIG. 15. Thus any perfusion temperature at or below 50C should be suitable for maintaining negligible thermal tissue damage. Volume of exposure scale changed so there is a crossover between 500 V/cm threshold and electric current at its scale of 0-40 A, as seen in FIG. 17. This crossover point, where benefits to reduced currents and thermal damage are maximized while volume of affected region exposure is maintained at a clinically level, has been shown to occur between perfusate temperatures of 30-35° C. Therefore, the optimum balance of ablation size gain with reduced electric current occurs in the perfusate temperature range of up to 35° C. The ablation volume notably increases with perfusate temperature, and thus the maximum perfusate temperature should be used, with a cap placed when thermal damage and electric current get too high. This ultimately occurs at the ambient probe growth rate of electric current and all thermally affected volumes increase at a faster rate than electric field exposure volume and minimum diameter, and must remain within reasonable and practical (actual clinically implemented) limits. Thus, within a temperature range of 5-60° C., it has been found that 20-50° C. would seem to be a ideal point for increasing the ablation size as much as possible while reducing arcing and thermal damage. Practically, however, 30-40° C. would also seem reasonable with noted benefit still present. This may be more practical as many hospitals have equipment that will raise the fluid temperature into that range. For perfusate temperatures less than 20° C., the minimum diameter is reduced by over 20%.

The method of using this system also includes the use of a specific set of IRE pulse treatment parameters. Traditional pulse parameters may include delivering between 70-100 pulses between each pair of electrodes, and then alternating between the various pairs, if more than 1 pair has been inserted. For example, these pulse parameters may include delivering multiple trains (or sets) of 70 pulses between each pair of monopolar probes, with a 1500V/cm of separation, and each pulse being up to 100 microseconds in length. Additionally, it is common to synchronize the delivery of the pulses with an ECG device, as described in more detail in U.S. Pat. No. 8,903,488, filed May 28, 2009, entitled SYSTEM AND METHOD FOR SYNCHRONIZING ENERGY DELIVERY TO THE CARDIAC RHYTHM, which is hereby incorporate by reference.

A problem in the art currently exists for traditional pulse parameters for IRE treatment, such as these pulse parameters may lead to rise in temperature adjacent to the electrodes and/or a rise in potential arcing which may lead to a system failure. The modulated or cycled pulse parameters of this invention are intended to reduce potentially dangerous or problematic temperature rise adjacent to the electrodes and reduce arcing potential.

The specific pulse parameters to be used with the above described system are intended to modulate or control the specific pulse parameters, thereby reducing the number of pulses and/or increase delays between pulses during treatment. For example, modulated pulse delivery may add deliberate pauses or delays between pulses to allow edema or tissue-scare effect normalization, electrolysis, gas dissolution, and/or ionic rebalancing. The method may provide a user with the ability to select preset pulse parameters, alter present pulse parameters, or provide for customization of pulse parameters. The advantage of modulating the pulse parameters will be to increase ablation volume, improve tumor response, decrease IRE-mitigating temperatures, allow tissue settling, and monitor or adjust pulse parameters in real-time during procedure.

Modulated pulse delivery may extend the duration in which a cell remains permeabilized. Increasing a cell's permeability reduces the cell membrane's ability to maintain a physical barrier between intracellular contents and the surrounding environment. Tissue regions experiencing sub-lethal electric fields will undergo pore alteration. Modulated pulse delivery enables advantages of maintaining cell poration for a longer period prior to re-insult, without costing additional procedural time. The effects of modulated pulse delivery may include, but are not limited to: (i) the tissue has the chance to return to baseline temperatures and electric conductivities before undergoing additional pulses; (2) secondary physiological responses, such as edemas, have opportunity to occur and distribute throughout the tissue; (3) improved pulse delivery without arcing or increasing arcing potential; and (4) extention of cell stress periods to increase lethal effect of pulses being delivered.

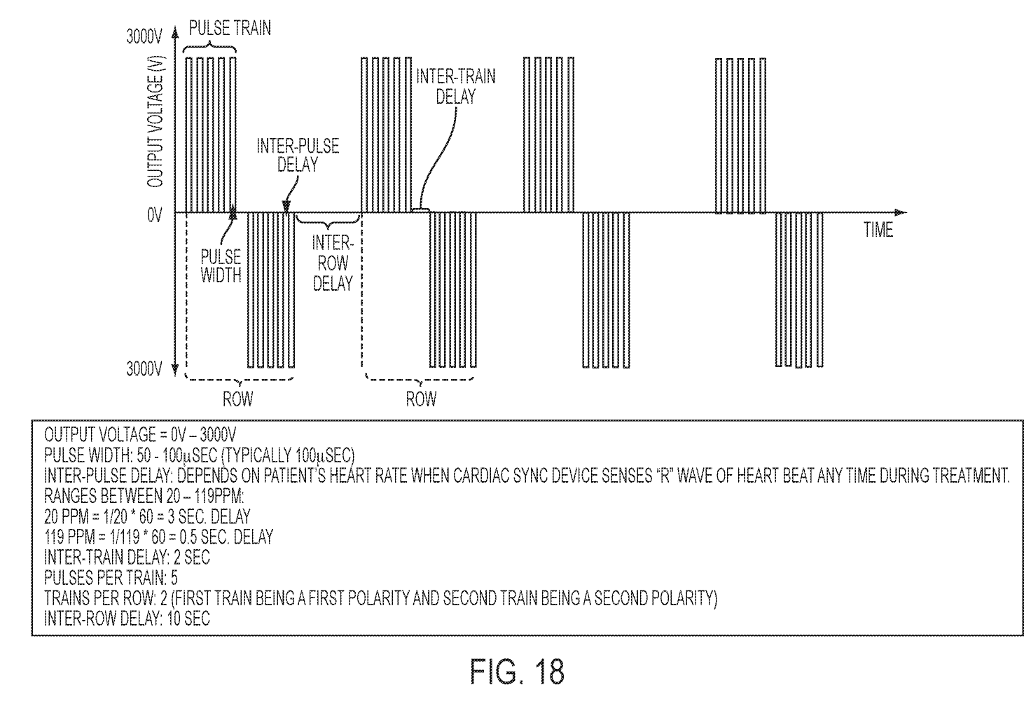

Referring to FIG. 18, in one embodiment the modulated pulse parameter timing algorithm to be used with this system includes a total of 400 pulses being delivered to the patient. The algorithm includes a first pulse train consisting of five single pulses. The voltage of each pulse may be up to 3000V. The pulse width may be up to 100 μsec, with an inter-pulse delay dependent on the patient's heart rhythm, but typically between 0.5 and 3.0 seconds. The first pulse train is of a first polarity, which may be either positive or negative. The second pulse train will follow the first pulse train after an inter-train delay of up to 2 seconds. The polarity of the second pulse train may be of a second polarity, and in this embodiment the second polarity will be the opposite of the first polarity. For example, if the first polarity of the first train is positive then the second polarity of the second train will be negative. The first and second trains combined equal one row of pulses. There is an inter-row delay of up to 10 seconds. After the inter-row delay, the second row will begin with a third train of five individual pulses. The third train of pulses will have a third polarity, and in this embodiment the third polarity is the same as the first polarity of the first train. The algorithm runs for a total of 400 pulses, which equals 40 total rows, and 80 total trains, with 40 trains in the first polarity and 40 trains in the second polarity.

Modulated or cycled pulse parameters may maintain more residual heating at low temperatures (<43° C.) but significantly reduce the volume exposed to higher temperatures as shown in the Table 3 below.

TABLE 3
Effects of modulated pulse parameters on residual heating.

| | Number of | Volume | Volume | Volume | Volume | Volume |
|---|---|---|---|---|---|---|
| Simulation | Pulses | T > 43 C. | T > 48 C. | T > 53 C. | T > 58 C. | T > 63 C. |
| Continuous | 80 | 4.54 cm | 0.78 cm | 0.34 cm | 0.145 cm | 0.027 cm |
| Pulses | | | | | | |
| Cycled | 80 | 5.28 cm | 0.53 cm | 0.213 cm | 0.061 cm | 0.002 cm |
| Pulses | | | | | | |
| Continuous | 100 | 5.07 cm | 0.89 cm | 0.40 cm | 0.17 cm | 0.040 cm |
| Pulses | | | | | | |
| Cycled | 100 | 6.15 cm | 0.58 cm | 0.23 cm | 0.07 cm | 0.0025 cm |
| Pulses | | | | | | |

An in vivo experiment was conducted to test for the optimal bipolar IRE pulse parameters to be used with a perfused system as described above. The cycled pulses include a delay as discussed below, whereas continuous pulses do not contain such delay.

Single electrode bipolar IRE was performed in 28 in-vivo pig livers (total of 78 ablations). First, effects of voltage (2,700-3,000 V), number of pulses, number repeated cycles (1-6), and pulse width (70-100 μsec) were studied. Next, electrical conductivity was altered by introduction of hypertonic and hypotonic fluids into the tissue using an open perfusion system Finally, effects of thermal stabilization were assessed using a closed perfusion system. Treatment effect was evaluated 2-3 hours post-IRE. Dimensions were compared and subjected to statistical analysis.

The study results demonstrated that by modifying multiple IRE parameters, one can achieve a clinically relevant benchmark of 3 cm short axis tissue ablation with a single bipolar probe. To obtain this result, the study delivered the IRE pulses over multiple cycles of application and coupling pulse delivery with altering tissue electrical conductivity by either systematically introducing hypotonic solution infusion or internally perfusing the electrode probe with perfusate, all of which are designed to increase voltage maximums and pulse lengths to the greatest possible without inducing over-current or electrical arcing issues.

First, the study examined the manipulation of IRE pulses without any perfusate. The study demonstrated that multiple cycles of pulses increased the diameter of the ablation to 2.9 cm. Yet, it was observed that for the bipolar configuration that this set of parameters increased system instability. Specifically, a much greater number of electrical pulse spikes are noted, which was attributed to greater electric arcing caused by the higher electrical fields encountered. Furthermore, it was demonstrated that the pulse parameters could result in enhancement of the ablation effect. Several of the tested tissue modifications resulted in an increased frequency of intense arcing and premature generator shut down. Particularly, the study increased IRE pulse length above the recommended 70 μsec to 100 μsec. While this modification did result in an increase in the short axis diameter of ablation effect from 2.6 cm to 2.9 cm, it was accompanied by increasing electrical spikes at the end of the IRE pulse. Although in an attempt to eliminate heat and gas build up surrounding the electrode by increasing the time between cycles of IRE application from 50 to 100 seconds, this alone could not eliminate the electrical instability. Summary of these results are shown in Table 4 below:

TABLE 4
Multiple repeat cycles with electrode tip exposure of 7 mm/8 mm of
insulation/7 mm of tip exposure:

| | | | Delay | | | | | |
|---|---|---|---|---|---|---|---|---|
| Voltage | Number | Pulse | Pulse | | System | Treatment | Treatment | Treatment |
| Range | of | Length | Duration | # of | Crash | Duration | Width | Length |
| (V) | Pulses | (μsec) | (sec) | trials | % | (min.) | (cm) | (cm) |
| 2,700 V | 90 | 70 | 50 | 5 | 0 | 13.2 ± 3.8 | 2.6 ± 0.3 | 4.5 ± 0.4 |
| 2,700 V | 50 | 100-70* | 100 | 10 | 40 | 19.2 ± 3.6 | 2.4 ± 0.1 | 4.6 ± 0.2 |
| 3,000 V | 50 | 100-70* | 100 | 10 | 50 | 18.4 ± 3.3 | 2.9 ± 0.1 | 5.0 ± 0.4 |

*starts with 100 μsec and gradually reduce to 70 usec to prevent arcing and system crash

Next, the study attempted and succeeded in increasing system stability by infusing hypotonic distilled water during IRE application. This change in electrical conductivity and increased stability of the system, with no arcing or crashes. Yet, there was a decrease in ablation size thus failing to meet a primary objective of creating large treatment zones. To account for these opposing effects, it was hypothesized that the perfusion of fluid in the tissue flushes out microbubbles created by the high intensity electrical field. Summary of these results are shown in Table 5 and Table 6 below:

TABLE 5
Summary of ablation sizes for high (100%) and low (10-25%)
concentration of saline infusion into tissue surrounding the electrode having
exposed tip 5-15 mm and insulation 5-8 mm:

| | | | Pulse | | System | | Treatment | | |
|---|---|---|---|---|---|---|---|---|---|
| Fluid | Voltage | # of | Length | | Crash | Rounds | Duration | Width | Length |
| Type | (V) | Pulses | (μsec) | N= | % | Complete | (min) | (cm) | (cm) |
| 100% | 2,700- | 50 | 100 | 3 | 100 | 1 | 7.0 ± 0.1 | 2.2 ± 0.2 | 4.6 ± 0.1 |
| Normal | 3,000 | | | | | | | | |
| Saline | | | | | | | | | |
| 10-25% | 3,000 | 50 | 100 | 3 | 66 | 2 | 11.3 ± 0.3 | 2.2 ± 0.2 | 4.7 ± 0.3 |
| Normal | | | | | | | | | |
| Saline | | | | | | | | | |

TABLE 6
Results of ablation sizes for infusion of distilled water (“DW”),
comparing constant flow vs. limited infusion into tissue surrounding the electrode
having exposed tip 5-15 mm and insulation 5-8 mm:

| | | | Pulse | | High | | Treatment | | |
|---|---|---|---|---|---|---|---|---|---|
| Fluid | Voltage | # of | Length | | Current | System | Duration | Width | Length |
| Type | (V) | Pulses | (μsec) | N= | % | Crash % | (min) | (cm) | (cm) |
| DW | 2,700- | 50 | 100 | 3 | 0 | 0 | 11.4 ± 3.4 | 2.3 ± 0.1 | 3.8 ± 1.1 |
| Constant | 3,000 | | | | | | | | |
| Flow* | | | | | | | | | |
| DW | 2,700- | 50 | 100 | 8 | 0 | 0 | 12.7 ± 2.3 | 2.7 ± 0.2 | 5.0 ± 0.1 |
| Constant | 3,000 | | | | | | | | |
| Flow | | | | | | | | | |
| DW | 2,700- | 50 | 100 | 4 | 75 | 25 | 13.1 ± 1.1 | 3.1 ± 0.3 | 5.1 ± 0.5 |
| Alternate | 3,000 | | | | | | | | |
| Flow** | | | | | | | | | |
| DW | 2,700- | 50 | 100 | 12 | 66.7 | 50 | 12.8 ± 0.2 | 3.1 ± 0.3 | 5.3 ± 0.6 |
| Alternate | 3,000 | | | | | | | | |
| Flow*** | | | | | | | | | |

*5 mm exposure and insulation
**Audible popping triggered
***Only while on for last 4 sets

Finally, the study tested an internally perfused electrode probe design that would reduce the inherent electrical conductivity rise of the tissue by mitigating tissue heating at the tissue-electrode interface. This strategy indeed did have the desired effect of permitting stable IRE application of sufficient duration and intensity to reliably produce 3 cm short-axis diameter treatment effect. The study found that the best results were seen when stabilizing the tissue properties with a warmer perfusate. Unlike known thermal ablation techniques, such as RF or microwave ablation, where cooler perfusate temperatures result in clinically better results, this study discovered that internal perfusion of the probe using body temperature perfusate resulted in clinically acceptable larger treatment zones. This likely results from an optimal balance between mitigating microbubble formation and/or excessive tissue electric conductivity rise to reduce likelihood of arcing, but not to the extent where it pronouncedly alters the electric field distribution to shrink one of the ablation dimensions. This is likely due to the redistribution in electric conductivity and thus lethal electric field distribution when the tissue nearest the electrode is ‘over-cooled’. Thus, optimal results seem to occur when perfusion is sufficient to control electrical conductivity rise enough to eliminate electrical arcing, but not so much that it dramatically alters the electric field distribution and treatment zone. Therefore, this study confirmed a key difference between IRE and thermally-dependent ablation modalities in that an electrode probe that is internally perfused with warm perfusate will treat significant targeted volumes without inducing protein denaturation due to temperature rise. Summary of these results are shown in Table 7 below:

TABLE 7
Results of ablation size for closed perfusion, comparing distilled water
at 4-10° C. vs. 37° C. vs. no fluid.

| | | Treatment | | | Ablation | Ablation |
|---|---|---|---|---|---|---|
| | Voltage | Duration | | System | Width | Length |
| Fluid Type | (V) | (min) | N= | Crash % | (cm) | (cm) |
| Cooled DW | 2,700- | 18-23 | 4 | 0 | 2.3 ± 0.1 | * |
| (4-10° C.) | 3,000 | | | | | |
| Warm DW | 2,700- | 18-23 | 4 | 0 | 3.1 ± 0.1 | 4.5 ± 1.3 |
| (37° C.) | 3,000 | | | | | |
| No Fluid | 2,700- | 18-20 | 3 | 66 | 30 ± 0.1 | 5.0 |
| | 3,000 | | | | | |

* system crashed and this data point was not recorded

Delivering 3,000 V at 70 μsec for a single 90 pulse cycle yielded 3.8±0.4×2.0±0.3 cm of ablation. Applying 6 cycles of energy increased the ablation to 4.5±0.4×2.6±0.3 cm (p<0.001). Further increasing pulse lengths to 100 μsec (6 cycles) further increased ablation to 5.0±0.4×2.9±0.3 cm (p<0.001), but resulted in electric spikes and system crashes in 40-50% of cases. Increasing tissue electrical conductivity via hypertonic solution instillation in surrounding tissues increased the frequency of generator crashes, whereas continuous instillation of distilled water eliminated this arcing phenomenon, but reduced ablation to 2.3±0.1 cm. Controlled instillation of distilled water when electrical arcing was suspected (using from audible popping as a trigger) produced ablations of 5.3±0.6×3.1±0.3 cm without crashes. Finally, 3.1±0.1 cm short-axis ablation was achieved without system crashes with internal electrode probe perfusion at 37° C. vs. 2.3±0.1 cm with 4-10° C. perfusion with no system crashes (p<0.001). This study confirms the potential utility of using a paradigm of IRE treatment based upon a single applicator bipolar electrode configuration. Most notably, it was demonstrated that by modifying is multiple IRE parameters, one can achieve a clinically relevant benchmark of 3 cm short axis tissue ablation with a single electrode probe insertion. The IRE paradigms to enable this include delivering the pulses over multiple cycles of application and coupling pulse delivery with altering tissue electrical conductivity by either systematically introducing hypotonic solution infusion or internally perfusing the electrode with body temperature perfusate, all of which are designed to enable increasing voltage and pulse length to the greatest possible without inducing over-current or electrical arcing issues.

The method of this system may also include the use of a treatment monitoring system customize pulse parameters in real-time during a treatment. The intent is to monitor the effects of the pulses being delivered to ensure the parameters are strong enough to achieve desired ablation sizes but also remain below key thresholds that will include exceeding 50 A (cause arcing) or raising temperature surrounding the electrodes above 43° C. This device may be used to treat a wide variety of tissue types and tissue parameters.

Examples of metrics to be measured may include, but are not limited to, low current which would indicate insufficient energy and that voltage may need to be increased; high current which would indicate that the user can lower voltage if current is approaching 50 A; current drifting higher which is a precursor to arcing and will indicate to the user to lower voltage to prevent unwanted arcs; an unstable current which is indicated by waveforms peaking near the end of the train; alerting the user to lengthen the pulse or reduce voltage; or satisfactory ablation which is when a particular electrode pair is identified as achieving satisfactory ablation and that the pair can be removed from the protocol thereby eliminating redundant pulses and saving time.

The method of a treatment monitoring system may include intraprocedural monitoring which will help assist the user in delivering ideal pulse parameters to maintain complete and effective pulse delivery when treating different tissue types as well as throughout the entire procedure as tissue parameters change. Examples of intraprocedural monitoring systems include the use of a Hall Effects probe to provide real-time electric current data and may be used to guide and inform procedures and parameters decisions. Another embodiment would be tracking a higher resolution understanding of procedure can be accomplished by breaking the pulse protocols down to delivery pulses in smaller quantities (such as 10-40 pulses per train rather than 70-100 pulses/train) at one time. Decisions by user to adjust the pulse parameters may be done prior to proceeding with the next pulse sequence.

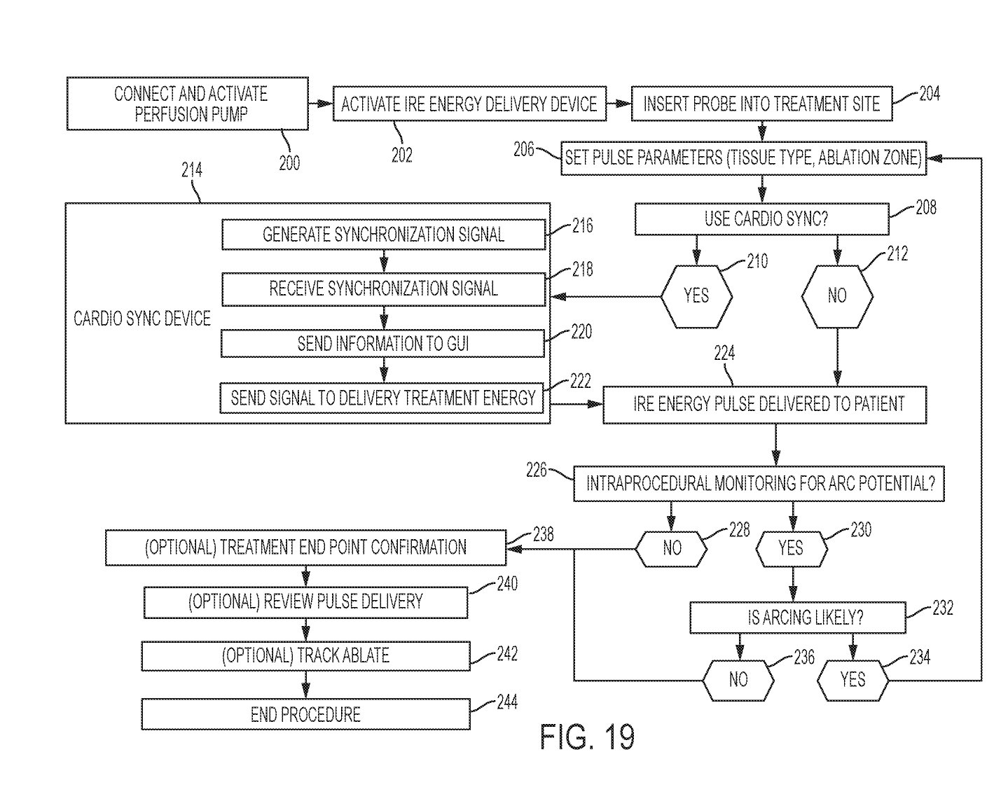

Referring now to FIG. 19, the steps for the method of using the system will be explained in detail. First, the user may prime the perfusion system 200 and connect the perfusion tubing, perfusion pump and any other perfusion components that may be required. The user may then activate the IRE energy delivery device 202 by powering on the various components of the system. After connecting the probe to the generator, the user may insert the probe into the treatment site 204, optionally using the imaging system for guidance. The GUI may then prompt the user to set specific pulse parameters 206 which may include, but are not limited to, the tissue type to be treated and the desired ablation zone. Based on the selected pulse parameters, the controller will automatically calculate the required parameters to achieve the desired settings. The GUI may next prompt the user to select if cardio sync 208 is to be used. If yes 210, the cardio sync device 214 will first generate a synchronization signal 216, then receive a synchronization signal 218, send information to the GUI 220, and then send the signal to delivery treatment energy 222. Alternatively, a test pulse may be sent to determine if the pulse parameters are satisfactory. If no cardio sync is to be used 212, or after the cardio sync device 214 has sent the signal to delivery treatment energy 222, the IRE energy pulses may be delivered to the patient 224. In certain embodiments of this method, it may be possible for intraprocedural monitoring for arc potential 226. If such monitoring is done 230, the system may monitor parameters to determine if an arc is likely to occur 232, and if it is 234 the GUI then may be triggered to go back and require the user to reset the pulse parameters 206. Conversely, if no 228 intraprocedural monitoring is being done or if arcing is not likely 236, then the method may optionally provide for a series of steps including, but not limited to, treatment end point confirmation 238, review of pulse delivery settings 240, and/or track ablation 242. Lastly, the procedure will end 244 and the method has been completed.

This device and method of use is intended to be used in various tissue types. The insertion of the probe 2 can be percutaneous, laparoscopic, endoscopic, as well as through natural orifices, including insertions related to orifice translumenal endoscopic surgery. One of ordinary skill in the art will recognize that other tissue types can be treated as well, including, but not limited to, digestive, skeletal, muscular, nervous, endocrine, circulatory, reproductive, vascular, integumentary, lymphatic, adipose, urinary, and soft tissue. The energy delivery probe 2 can be suitable for treatment of conditions for various tissues, volumes, sizes and locations, including small to medium sized tissue volumes, and tissue volumes that are in close proximity to other non-targeted structures, such as, but not limited to, neuronal structures, vascular structures, duct structures, and collagen-rich structures. Non-limiting examples of tissue masses to which the devices of the present application are applicable include benign tissue masses such as benign prostate hyperplasia (BPH) and uterine fibroids, as well as benign or malignant masses such as cancers and tumors of various tissue types, including, but not limited to, breast, brain, prostate, uterine, lung, liver, kidney, brain, head/neck, bone, stomach, colon, and pancreas. The method can also be used to target singly or in combination tissues that are benign, malignant, cancerous, neoplastic, preneoplastic, or tumorous.

One example of infected tissue that can be treated using IRE and this system is infected bone, or osteomyelitis. Bone infections can be extremely difficult to treat. Typically, bone infections can be treated using surgical procedures. The bone can be accessed by variety of procedures, such as through the skin. After the bone is surgically cleaned out, the remaining bone defect(s) is treated with a large dose of antibiotics via a non-resorbable bone cement to eradicate any bacterial cells in the bone and bloodstream. After this, subsequent surgery is required for removal and replacement with a bone graft or an absorbable mix of synthetic bone substitute. After a bone cleaning and replacements, the bone is typically not strong enough to bear weight. Bone rebuilding techniques can involve bone grafting or bone transport. Antibiotic treatment is then administered through an intravenous catheter. These treatment procedures have the attendant disadvantages mentioned above. Instead of using the above-described extensive, painful, and expensive procedures, this system may use IRE to treat bone infection. In one aspect, sufficient electrical pulse parameters can be selected, as described herein above, to irreversibly electroporate infected cells that are present within or along bone. In one aspect, the single bipolar probe described herein can be inserted into a target tissue surrounding an infected bone, and sufficient electrical pulse parameters could be selected to adequately irreversibly electroporate an infected bone mass. In one embodiment, an outer layer of bone could be treated to remove infected cells. When infected tissue of a bone is irreversibly electroporated, such target bone tissue could include muscle and/or vessels which could be acutely necrosed. However, in time, the critical cellular and/or vascular structures could grow back so that no long term harmful consequences would occur.

1. A medical device for ablating tissue cells in a treatment region by irreversible electroporation without thermally damaging the tissue cells, comprising:
a temperature: controlled perfusate;
an electrode probe having a perfusate channel for receiving the temperature controlling perfusate and at least two electrodes adapted to apply irreversible electroporation (IRE) pulses to the tissue cells in the treatment region;
a control device for controlling the IRE pulses to the at least two electrodes and operable to provide the temperature controlling perfusate to the perfusate channel of the probe to maintain the temperature of the tissue cells between 20 degrees Celsius and 50 degrees Celsius.

2. The medical device of claim 1, wherein the temperature controlling perfusate controls an electrical conductivity rise in the tissue cells sufficiently to eliminate electrical arcing, but without significantly altering the electric field distribution in the treatment zone.

3. The medical device of claim 1, wherein the control device provides the temperature controlling perfusate to the perfusate channel to maintain the temperature of the tissue cells at between 30 degrees Celsius and 45 degrees Celsius.

4. The medical device of claim 1, wherein the electrode probe includes a temperature sensor that measures the temperature of the tissue cells and the control circuit adjusts the amount of the temperature controlling perfusate delivered to the perfusate channel in real-time based on the measured temperature.

5. The medical device of claim 1, further comprising a power distribution unit.

6. The medical device of claim 1, further comprising a pump coupled to the control device, wherein the control device controls the pump to vary the flow rate of the temperature controlling perfusate.

7. The medical device of claim 1, further comprising a pulse generator capable of generating the IRE pulses wherein the IRE pulses in one train between the two electrodes have a first polarity and the IRE pulses in an adjacent train have a second polarity opposite from the first polarity.

8. The medical device of claim 1, wherein the control device monitors the current flow through the at least one electrode and provides the temperature controlling perfusate to the perfusate channel based on the monitored current flow.

9. The medical device of claim 1, wherein the control device monitors the current flow through the at least one electrode and provides the temperature controlling perfusate to the perfusate channel based on the rate of change of the current.

10. The medical device of claim 6, wherein the electrode probe includes fluid ports along its distal end, wherein the temperature controlling perfusate is introduced into the tissue cells through the fluid ports.

11. The medical device of claim 1, wherein the control device calculates tissue conductivity based on the current flow through the at least one electrode.

12. The medical device of claim 11, wherein the control device applies a test pulse through the electrode and calculates the tissue conductivity based on the current flow from the applied test pulse.

13. The medical device of claim 1, further comprising a temperature sensor that senses the temperature of the target region, wherein the control device calculates tissue conductivity based on the sensed temperature.

14. The medical device of claim 1, wherein the control device controls the flow of the temperature controlling perfusate through the perfusate channel based on at least one of the number of IRE signals, current or the amount of power applied to the target region.

15. The medical device of claim 1, further comprising a memory that stores at least one electrical parameter for a plurality of tissue types and the control device controls the flow of the temperature controlling perfusate through the perfusate channel based on the at least one electrical parameter for the type of tissue cells being treated.

16. The medical device of claim 1, further comprising:
a pumping device that controls the flow rate of the temperature controlling perfusate through an source tube and a return tube;
wherein the pumping device is controlled by the control unit.

17. A method of ablating tissue cells in a treatment region by irreversible electroporation without thermally damaging the tissue cells, comprising:
applying irreversible electroporation (IRE) signals to the tissue cells in the treatment region through at least one electrode of an electrode probe;
providing a temperature controlling perfusate to a perfusate channel of the electrode probe to maintain the temperature of the tissue cells at 45 degrees Celsius or less.

18. The method of claim 17, wherein the step of providing includes providing temperature controlling perfusate to the perfusate channel to maintain the temperature of the tissue cells at body temperature.

19. The method of claim 17, further comprising:
controlling an electrical conductivity rise in the tissue cells sufficiently to eliminate electrical arcing with the temperature controlling perfusate; the step of eliminating significantly altering the electric field distribution and treatment zone.

20. A medical device for ablating tissue cells in a treatment region by irreversible electroporation without thermally damaging the tissue cells, comprising:
an electrode probe having first and second spaced apart electrodes;
a pulse generator that generates IRE pulses as follows: a first row of pulses consisting of a first pulse train and a second pulse train, the first pulse train consisting of at least five individual pulses, the first pulse train having a first polarity, an inter-train delay of at least 2 second, the second pulse train consisting of at least five individual pulse second pulse train having a second polarity that is the opposite of the first polarity, an me r delay of up to at least 10 second, a second row of pulses consisting of a third pulse train and a fourth pulse train.
